(19) United States
(12) Patent Application Publication
(10) Pub. No.: US 2019/0231704 A1
(43) Pub. Date: Aug. 1, 2019

(54) COMPOSITIONS FOR ENHANCED UPTAKE BY MACROPHAGES AND METHODS OF USE THEREOF

(71) Applicants: Yale University, New Haven, CT (US); UNIVERSITY OF SOUTHERN CALIFORNIA, Los Angeles, CA (US)

(72) Inventors: Tarek Fahmy, Middlefield, CT (US); Terrence C. Town, Los Angeles, CA (US); Tara M. Weitz, Los Angeles, CA (US)

(21) Appl. No.: 16/336,453

(22) PCT Filed: Aug. 22, 2017
(86) PCT No.: PCT/US2017/048015

§ 371 Date: Mar. 25, 2019

Related U.S. Application Data
(60) Provisional application No. 62/399,175, filed on Sep. 23, 2016.
(60) Provisional application No. 62/421,824, filed on Nov. 14, 2016.
(60) Provisional application No. 62/489,227, filed on Apr. 24, 2017.

(51) Int. Cl. A61K 9/51; A61K 9/127; A61K 47/69; A61K 47/68; A61K 49/18; A61P 25/28; A61K 31/4439

(52) U.S. Cl. CPC A61K 9/5153; A61K 9/1271; A61K 47/6937; A61K 31/4439; A61K 49/1827; A61P 25/28; A61K 47/6849

(57) ABSTRACT

Negatively charged nanoparticulate compositions are used to deliver therapeutic, prophylactic or diagnostic agents to macrophages or other phagocytic cells in the brain and central nervous system. The negative charge of the nanoparticles increases circulation, increases internalization by macrophage or other phagocytic cells, increases release within the macrophage or other phagocytic cells, or a combination thereof, relative to charge-neutral or charge-positive nanoparticles.

### CROSS-REFERENCE TO RELATED APPLICATIONS

This application claims benefit of U.S. Provisional Application No. 62/399,175 filed Sep. 23, 2016, U.S. Provisional Application No. 62/421,824 filed Nov. 14, 2016, and U.S. Provisional Application No. 62/489,227 filed Apr. 24, 2017, hereby incorporated herein by reference in their entirety.

### STATEMENT REGARDING FEDERALLY SPONSORED RESEARCH OR DEVELOPMENT

This invention was made with government support under R01 NS076794 awarded by the National Institutes of Health. The government has certain rights in the invention.

### FIELD OF THE INVENTION

The field of the invention is generally related to nanoparticulate compositions designed for enhanced uptake by immune cells of the monocytic lineage (e.g., macrophages or microglia or Kupffer cells or monocytes or mononuclear phagocytes or dendritic cells or other phagocytic cells), and methods of using particle-loaded macrophage to traffic therapeutic, diagnostic, and other active agents to sites of interest including inflammation, degeneration, infection, and cancer.

### BACKGROUND OF THE INVENTION

Although delivery of active agent has been improved by nanotechnology, many active agents still do not reach the target location in an effective amount to improve the symptoms of a sick subject. This is particularly true in the brain, where drug delivery into the brain is difficult because of fast metabolism and/or rapid clearance, as well as poor permeation through the blood-brain barrier (BBB) (Pardridge, J Cereb Blood Flow Metab, 32: 959-972 (2012)).

Therefore, there remains a need to further improve delivery of active agents to sites of disease and disorder within the body, including the brain, and to do so in an effective amount to improve the health of the subject.

It is an object of the invention to provide compositions and methods of use thereof to improve delivery of active agents to the brain and CNS and particulate matter such as bacteria, viruses, parasites, cell debris, and peptide aggregates.

### SUMMARY OF THE INVENTION

Immune cells of the monocytic lineage (e.g., macrophages or microglia or Kupffer cells or monocytes or mononuclear phagocytes or dendritic cells or other phagocytic cells) are “first responders” in many disease states. They are the primary immune cells that respond to an infection, wound or any bodily injury. As such, macrophages alert the immune system (both innate and adaptive) to the nature of dysfunction. It has been discovered that while positively charged nanoparticles (NP) are non-discriminately taken up by various cells and organs, negatively charged NP, particularly in the presence of cations, are initially repelled or slightly positively modulated by cationic species and then efficiently taken up by macrophages. The negatively charged particles either alter the phenotypic characteristics of the endocytosing cell or are later efficiently exocytosed from the cells, enabling them to discharge the intact nanoparticles at a location distal to where they were initial internalized. The nanoparticles can be internalized by target cells in the distal location, degrade and thus release their cargo into the endocytosing cell, or the microenvironment of the distal location, or a combination thereof. The kinetics of circulation, internalization, and exocytosis, which is dependent at least in-part on the surface charge of the nanoparticles, is important for using macrophage and other phagocytotic cells in this way as carriers for transporting nanoparticulate cargo to sites of disease and inflammation. Thus, circulation time for efficient uptake by phagocytic cells and uptake time are critical parameters governing how far nanoparticles can distribute and with what efficiency the cargo is delivered to where it needs to go.

The nanoparticulate compositions are efficiently taken up by macrophage and other phagocytic cells are provided. Macrophages are potent phagocytes and are adept at clearing particulate matter, such as bacteria, viruses, parasites, cell debris and protein aggregates, methods of using the nanoparticle-loaded macrophages as carriers (i.e., as “Trojan horses”) to traffic one or more active agents encapsulated in the nanoparticle are also provided. In one embodiment, nanoparticles are used to specifically deliver therapeutics to macrophages or other phagocytic cells, thereby modulating macrophage activity. Exemplary agents include, for example, therapeutic drugs (e.g., immunomodulatory drugs), invasive and non-invasive imaging agents, etc. Thus, the methods can be therapeutic or diagnostic in nature.

For example, a composition for using macrophage or other phagocytic cells as nanoparticle-carriers can include active agent-encapsulated negatively charged nanoparticles that can be internalized by macrophage or other phagocytic cells in vivo, trafficked by the cells to a distal site, and exocytosed by the cells in an effective amount to have a therapeutic or diagnostic effect in the microenvironment of the distal location. In another embodiment, a composition encompassing negatively charged nanoparticles is used to deliver an active agent to macrophages or other phagocytic cells, thereby modulating the activation state or changing the phenotype of the macrophage or other phagocytic cell. Exemplary agents include immunomodulatory drugs, such as, for example, TGF-beta inhibitors. The negative charge of the nanoparticles can be effective to (1) increase circulation in the subject to whom the nanoparticles are administered following systemic administration, (2) increase internalization by macrophage or other phagocytic cells, (3) increase exocytosis of the macrophage or other phagocytic cells, or (4) a combination thereof, relative to charge-neutral or charge-positive nanoparticles. In some embodiments, the zeta potential of the nanoparticles is between about −100 mV and about 0 mV, preferably between about −20 mV and about −1 mV.

The nanoparticles can be formed of polymers such as pegylated polyesters like lactide-co-glycolide—PEG triblock polymers or protein-based aggregates, liposomes, or multilamellar vesicles. For example, the nanoparticles can be formed of polymers such as the lactide-co-glycolide—PEG triblock polymer having the structure A-X where A is a hydrophobic molecule or hydrophobic polymer, and X is a terminal moiety that imparts a negative charge to the particle. The nanoparticles can be polymeric nanoparticles including the structure A-B-X where A is a hydrophobic molecule or hydrophobic polymer, B is a hydrophilic molecule or hydrophilic polymer, and X is a terminal moiety that imparts a negative charge. In some embodiments, the nanoparticles are liposomes including an anionic lipid; a negatively charged moiety attached to a cationic, neutral lipid, an anionic lipid, and/or to a linker such as PEG; or a combination thereof. In preferred embodiments, the terminal moiety is an acidic group or an anionic group pendant on a hydrophilic group (PEG). Acidic groups include, for example, carboxylic acids, protonated sulfates, protonated sulfonates, protonated phosphates, singly- or doubly protonated phosphonates, and singly- or doubly protonated hydroxamate. Anionic groups include, for example, carboxylates, sulfates, sulfonates, singly- or doubly deprotonated phosphate, singly- or doubly deprotonated phosphonate, and hydroxamate. In some embodiments, the nanoparticles include a macrophage targeting moiety. In a specific embodiment the nanoparticles include a biodegradable polyester or polyanhydride such as poly(lactic acid), poly(glycolic acid), and poly(lactic-co-glycolic acid) conjugated to PEG conjugated to a negatively charged terminal moiety such as COOH.

The composition can include a cation in an effective amount to reduce the negative charge of the particles. The cation can be, for example, Ca²⁺ or Mg²⁺.

Exemplary therapeutic, diagnostic or prophylactic agents include, but are not limited to, small molecules (having a molecular weight under 2000 D, more preferably less than 1200 D, most preferably 1000 D or less), synthetic drugs, peptides (including cyclic peptides), polypeptides, proteins, nucleic acids, synthetic or natural inorganic or organic molecules, and imaging agents. In particular embodiments, the agent is an immunomodulator, an anti-inflammatory agent, or an imaging agent. In a specific embodiment the active agent is a TGF-β inhibitor.

Methods of treatment typically include administering to a subject an effective amount of the agent-encapsulated negatively charged nanoparticle composition. In some embodiments, the subject has a neurodegenerative disease, a proliferation disorder such as cancer, an inflammatory disorder, or an autoimmune disease. In particular embodiments, the subject has a disease or disorder characterized by misfolded protein aggregates and the active agent is a TGF-β inhibitor. Examples of such diseases include Alzheimer's disease, Parkinson's disease, amyotrophic lateral sclerosis, frontotemporal dementia, Lewy body dementia, traumatic brain injury, Type II diabetes, atherosclerosis or another manifestation of cardiovascular disease, or autoimmune disease. In another embodiment, the subject has a form of cancer, such as a brain cancer like medulloblastoma, glioblastoma, astrocytoma or glioblastoma multiforme. The composition can be administered systemically.

An exemplary method of imaging a subject can include (a) administering to a subject the active agent-encapsulated negatively charged nanoparticle composition, wherein the active agent is an imaging agent, and (b) acquiring at least one image of at least a portion of the subject.

### BRIEF DESCRIPTION OF THE DRAWINGS

FIG. 1 is a bar graph showing the effect of magnesium (Mg²⁺) ion concentration on macrophage internalization of Coumarin 6-loaded (500 μg/ml) (Fluorescence intensity (pixels)) PLGA-PEG-OMe and PLGA-PEG-COOH nanoparticles (24 hours).

FIG. 2 is a bar graph showing the effect of calcium (Ca²⁺) ion concentration on macrophage internalization of Coumarin 6-loaded (250 μg/ml) (Fluorescence intensity (pixels)) PLGA-PEG-OMe and PLGA-PEG-COOH nanoparticles (48 hours).

FIG. 3 is a bar graph showing the effect of magnesium (Mg²⁺) ion concentration on macrophage internalization of Coumarin 6-loaded (250 μg/ml) (Fluorescence intensity (pixels)) PLGA-PEG-OMe and PLGA-PEG-COOH nanoparticles (48 hours).

FIG. 4 is a bar graph showing the effect of cell number on macrophage internalization of Coumarin 6-loaded (250 μg/ml) (Fluorescence intensity (pixels)) PLGA-PEG-OMe and PLGA-PEG-COOH nanoparticles.

The experiments illustrated in FIGS. 1-4 utilized 1×10⁵ cells per well.

FIGS. 5A-5B are bar graphs showing the effect of nanoparticle end group on macrophage uptake (5A) and nanoparticle charge (5B). FIG. 5C is a bar graph showing the effect of the combination of nanoparticle end group and magnesium (Mg2+) concentration on macrophage uptake.

FIGS. 6A-6C are bar graphs showing the impact of nanoparticle charge (-Ome (neutral) in 6A, —NH2 (positive) in 6B, and —COOH (negative) in 6C) on biodistribution to the heart, kidney, liver, lung, and spleen at 1, 6, and 24 hours.

FIGS. 6D-6F are bar graphs showing the impact of nanoparticle end group (-Ome (neutral), —NH2 (positive), and —COOH (negative)) on uptake in liver (6D), blood (6E), and tumor (6F).

FIG. 7A is a bar graph showing the effect of negative charge chelating EDTA on macrophage internalization of PLGA-PEG-Ome and PLGA-PEG-COOH nanoparticles. FIG. 7B is an illustration of the proposed effect of cations on nanoparticles with negatively charged end groups. FIG. 7C is an illustration of a proposed mechanism of negatively charged nanoparticle internalization by negatively charged cells such as macrophage.

FIG. 8 is a bar graph comparing macrophage internalization of PLGA nanoparticles and lapidated nanoparticles.

FIG. 9 is a dot plot showing the effect of concentration and macrophage targeting on the internalization of nanoparticles.

FIGS. 10A-10B are bar graphs showing the effect of nanoparticle internalization on macrophage function (DIL-labeled LDL update measured as mean fluorescence intensity (MFI)) of B3Z T cells (negative control), untreated macrophage, and macrophage targeted with various concentrations of anti-F4/80 antibody (10A) and anti-CD204 antibody (10B).

FIGS. 11A-11D are bar graphs showing the relative biodistribution (p/s/cm²/sr/gm tissue (1×10⁹)) of Macrophage-loaded Nanoparticles and free nanoparticles in the heart, lung, spleen, pancreas, kidney, brain, and liver in vivo, one (11A), four (11B), eight (11C), and twenty-four (11D) after intravenous administration.

FIGS. 12A-12G are line graphs showing the relative biodistribution (p/s/cm²/sr (1×10⁹)) of Macrophage-loaded Nanoparticles and free nanoparticles in the heart (12A), lung (12B), spleen (12C), pancreas (12D), kidney (12E), brain (12F), and liver (12G) in vivo over the first 24 hours after intravenous administration.

FIG. 13 is a curve showing the pharmacokinetics of Macrophage-loaded Nanoparticles (M-NP) and free nanoparticles (NP) in the blood over the first 24 hours after intravenous administration.

FIG. 14A is a schematic of macrophage-targeting nanoparticles. These ˜150 nm particles deliver Coumarin-6 or superparamagnetic iron oxide (SPIO) and TGF-beta-Smad 2/3 signaling inhibitor SB505124 to peripheral macrophages. SB505124 is the unencapsulated inhibitor. SB1947 is SB505124 loaded into nanoparticles. FIGS. 14B and 14C are graphs showing selective drug delivery to peripheral macrophages in vitro using nanoparticles (14B, uptake; 14C, release). Addition of the PEG moiety increases PLGA nanoparticle bioavailability to cultured rat peripheral monocytes.

FIGS. 15A-15D are graphs showing tissue distribution over time following administration (15A, one; 15B, four; 15C, eight, and 15D, 24 hours after administration). FIG. 15E is a bar graph showing amyloid-β phagocytosis (quantification of microglial phagosomes containing Aβ ) in TgF344-AD rat brains after nanoparticle delivery of SB1947. Data are mean±SEM (n=6 cells) for mononuclear phagocytes distant from plaques (1) or associated with plaques (2). **p<0.01; by Student's t-test. FIG. 15F and 15G are bar graphs showing quantitative cell-specific analysis indicating Coumarin-6 signal is found in 10-35% of cells expressing Iba1, CD11b, or MHCII and not in cells expressing MAP2, GFAP, or Olig4 (ND=not detected) (15F) and that the vast majority of Coumarin-6-positive cells (80-90%) associate closely with amyloid plaques (15G).

FIGS. 16A-16C are graphs showing that macrophages containing PEG-PGLA nanoparticles migrate to the CNS and are effective in reversing Abeta and tau aggregation and plaque formation.

FIG. 17 a bar graph showing pre-synaptic density (synaptophysin staining) of wildtype (WT untreated), Tg-F334-AD (untreated), and Tg-F334-AD (nanoparticle-loaded SB1947 (“nano-SB1947”)).

FIGS. 18A and 18B show that treatment of AD model rats with nano-SB1947 reverse behavioral defects in Tg-F344-AD rats according to Barnes Maze Test (18A) and Novel Object Recognition (18B).

FIG. 19A and 19B are bar graphs showing quantitation of Iba1+ mononuclear phagocyte volume occupied by LAMP1+ phagolysosomes (19A) and quantitation of intracellular LAMP1+ phagolysosomal volume occupied by 4G8+ (19B). Values are based on ratio of signal (voxels) above threshold that are colocalized. Data are shown as mean±standard error of the mean (n=6 cells) for mononuclear phagocytes distant from plaques (1), or associated with plaques (2). **p<0.001 by student's t-test.

FIG. 20A and 20B are bar graphs showing quantitation of Iba1+ mononuclear phagocyte volume occupied by CD68+ phagolysosomes (20A) and quantitation of intracellular CD68+ phagolysosomal volume occupied by OC+ Aβ. Values are based on ratio of signal (voxels) above threshold that are colocalized. Data are shown as mean±standard error of the mean (n=6 cells) for mononuclear phagocytes distant from plaques (1), or associated with plaques (2). **p<0.01 by student's t-test.

### DETAILED DESCRIPTION OF THE INVENTION

### I. Definitions

“Small molecule,” as used herein, refers to molecules with a molecular weight of less than about 2000 g/mol, more preferably less than about 1500 g/mol, most preferably less than about 1200 g/mol.

“Nanoparticle”, as used herein, generally refers to a particle having a diameter from about 10 nm up to, but not including, about 1 micron, preferably from 100 nm to about 1 micron. The particles can have any shape. Nanoparticles having a spherical shape are generally referred to as “nanospheres”.

“Molecular weight” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% of the distribution lies within 15% of the median particle size, more preferably within 10% of the median particle size, most preferably within 5% of the median particle size.

“Active Agent”, as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

“Negatively charged moiety,” refers to a functional group that imparts a negative charge to a molecule, particle, or other chemical group to which it is attached, covalently or non-covalently. Preferably, the negatively charged moiety is covalently attached to the molecule, particle, or chemical group. As used herein, “negatively charged moiety” can be an acidic group or an anionic group.

“Acidic group” refers to a functional group that is capable of donating protons or accepting a lone pair of electrons.

“Anionic group,” as used herein, refers to a functional group that is the salt of an acidic group. An anionic group can be formed from the deprotonation of an acidic group, or as in the case of boronic acid, from reacting with a Lewis base such as water, hydroxyl group, thiol group, or amino group. It should be noted that in solution, the acidic group and the anionic group generally exist in equilibrium, with the concentration of either group dependent on the pH of the solution.

### II. Compositions

It has been discovered negatively charged nanoparticles are more efficiently taken up by macrophages through cation-mediated complexation prior to macrophage uptake, especially as compared to uncharged or positively charged nanoparticles.

A. Negatively Charged Nanoparticles

In some embodiments, the charge of particles is determined by the zeta potential. The zeta potential of the nanoparticles is typically between about −100 mV and about 0 mV; between about −100 mV and about −2.5 mV; between about −100 mV and about −5 mV; between about −100 mV and about −7.5 mV; between about −100 mV and about −10 mV; between about −75 mV and about 0 mV; between about −75 mV and about −2.5 mV; between about −75 mV and about −5 mV; between about −75 mV and about −7.5 mV; between about −75 mV and about −10 mV; between about −50 mV and about 0 mV; between about −50 mV and about −2.5 mV; between about −50 mV and about −5 mV; between about −50 mV and about −7.5 mV; between about −50 mV and about −10 mV; between about −25 mV and about 0 mV; between about −25 mV and about −2.5 mV; between about −25 mV and about −5 mV; between about −25 mV and about −7.5 mV; between about −25 mV and about −10 mV; between about −20 mV and about 0 mV; between about −20 mV and about −2.5 mV; between about −20 mV and about −5 mV; between about −20 mV and about −7.5 mV; between about −20 mV and about −10 mV; between about −15 mV and about 0 mV; between about −15 mV and about −2.5 mV; between about −15 mV and about −5 mV; between about −15 mV and about −7.5 mV; between about −15 mV and about −10 mV; between about −10 mV and about 0 mV; between about −10 mV and about −2.5 mV; between about −10 mV and about −5 mV; between about −10 mV and about −7.5 mV; between about −7.5 mV and about 0 mV; between about −7.5 mV and about −2.5 mV; between about −7.5 mV and about −5 mV; between about −5 mV and about 0 mV; between about −5 mV and about −2.5 mV; or between about −2.5 mV and about 0 mV.

In the most preferred embodiments, the particles are nanoparticles, for example, 10 nm up to, but not including, about 1 micron, with a preferred range of less than 400 nm and greater than 40-60 nm. However, it will be appreciated that in some embodiments, and for some uses, the particles can be smaller or larger (e.g., microparticles, etc.). The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In preferred embodiments the nanoparticles have a diameter less than 400 nm, less than 300 nm, or less than 200 nm. The average diameters of the nanoparticles are typically between about 50 nm and about 500 nm, preferably between about 50 nm and about 350 nm, most preferably between about 100 nm and 300 nm. In some embodiments, the average diameters of the nanoparticles are about 100 nm.

1. Polymeric Nanoparticles

In preferred embodiment, the particles are polymeric nanoparticles. The nanoparticles can be formed of poly lactide-co-glycolide class with PEG as a pendant moiety, pegylated liposomes, or protein aggregates. In the most preferred embodiments, the particles are polyester-based polymeric nanoparticles.

a. Polymers

The polymers are preferably biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

The polymers can be polyesters or polyesters conjugated to or modified with one or more polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone, blends, and copolymers thereof.

The polyesters are preferably polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; and poly(orthoesters). The preferred hydrophobic polymer is poly(lactic acid), poly(glycolic acid), or poly(lactic acid-co-glycolic acid).

The nanoparticles can contain one or a mixture of two or more polyesters. The nanoparticles may contain other entities such as stabilizers, surfactants, or lipids. The nanoparticles may contain a first polymer having a terminal moiety and a second polymer not having the terminal moiety. By adjusting the ratio of the polymers with and without a terminal moiety, the density of the terminal moiety on the exterior of the particle can be adjusted.

The nanoparticles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In preferred embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both.

The nanoparticles can contain an amphiphilic polymer having a hydrophobic end, a hydrophilic end, and a terminal moiety attached to the hydrophilic end. In some embodiments the amphiphilic macromolecule is a block copolymer having a hydrophobic polymer block, a hydrophilic polymer block covalently coupled to the hydrophobic polymer block, and a terminal moiety covalently coupled to the hydrophilic polymer block. For example, the amphiphilic polymer can have a conjugate having the structure A-B-X where A is a hydrophobic molecule or hydrophobic polymer, preferably a hydrophobic polymer, B is a hydrophilic molecule or hydrophilic polymer, preferably a hydrophilic polymer, and X is a terminal moiety. Preferred amphiphilic polymers include those where A is a hydrophobic biodegradable polymer, B is a hydrophilic polymer, such as PEG, and X is a terminal moiety that imparts a negative charge to the particle.

In some embodiments the nanoparticles contain a first amphiphilic polymer having a hydrophobic polymer block, a hydrophilic polymer block, and terminal moiety conjugated to the hydrophilic polymer block; and a second amphiphilic polymer having a hydrophobic polymer block and a hydrophilic polymer block but without the terminal moiety. The hydrophobic polymer block of the first amphiphilic polymer and the hydrophobic polymer block of the second amphiphilic polymer may be the same or different. Likewise, the hydrophilic polymer block of the first amphiphilic polymer and the hydrophilic polymer block of the second amphiphilic polymer may be the same or different.

In some embodiments the nanoparticle contains a first amphiphilic polymer having the structure A-B-X as described above and a second amphiphilic polymer having the structure A-B, where A and B in the second amphiphilic macromolecule are chosen independently from the A and B in the first amphiphilic macromolecule, although they may be the same.

In some embodiments, the nanoparticle contains a polymer having the structure A-X where A is a hydrophobic molecule or hydrophobic polymer, preferably a hydrophobic polymer, and X is a terminal moiety that imparts a negative charge to the particle. Preferred polymers include those where A is a hydrophobic biodegradable polymer, and X is a terminal moiety that imparts a negative charge to the particle. Preferably, A is biodegradable.

In some embodiments, the nanoparticle contains a first polymer having the structure A-X where A is a hydrophobic molecule or hydrophobic polymer, preferably a hydrophobic polymer, and X is a terminal moiety that imparts a negative charge to the particle; and second amphiphilic polymer having a hydrophobic polymer block and a hydrophilic polymer block but without the terminal moiety. Preferably, A, the hydrophobic polymer block of the amphiphilic polymer, or both, are biodegradable.

In particularly preferred embodiments, the nanoparticle contains biodegradable polyesters or polyanhydrides. The nanoparticles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(ε-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyalkylene oxides such as polyethylene glycol (PEG) (straight or branched chain) or a polyalkylene oxide block copolymer such as PLURONIC® and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The nanoparticles can also contain one or more polymer conjugates containing end-to-end linkages between the polymer and a terminal moiety. For example, a modified polymer can be a PLGA-PEG-peptide block polymer.

b. Methods of Making Particles

Methods of making polymeric particles are known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

The polymeric nanoparticles are typically formed using an emulsion process, single or double, using an aqueous and a non-aqueous solvent. Typically, the nanoparticles contain a minimal amount of the non-aqueous solvent after solvent removal.

In some embodiments, nanoparticles are prepared using emulsion solvent evaporation method. A polymeric material is dissolved in a water immiscible organic solvent and mixed with a drug solution or a combination of drug solutions. The water immiscible organic solvent is preferably a GRAS ingredient such as chloroform, dichloromethane, and acyl acetate. The drug can be dissolved in, but is not limited to, one or a plurality of the following: acetone, ethanol, methanol, isopropyl alcohol, acetonitrile and Dimethyl sulfoxide (DMSO). An aqueous solution is then added into the resulting mixture solution to yield emulsion solution by emulsification. The emulsification technique can be, but not limited to, probe sonication or homogenization through a homogenizer.

In another embodiment, nanoparticles are prepared using nanoprecipitation methods or microfluidic devices. A polymeric material is mixed with a drug or drug combinations in a water miscible organic solvent. The water miscible organic solvent can be one or more of the following: acetone, ethanol, methanol, isopropyl alcohol, acetonitrile and Dimethyl sulfoxide (DMSO). The resulting mixture solution is then added to an aqueous solution to yield nanoparticle solution. The agents may be associated with the surface of, encapsulated within, surrounded by, and/or distributed throughout the polymeric matrix of the particles.

In another embodiment, nanoparticles are prepared by the self-assembly of the amphiphilic polymers, optionally including hydrophilic and/or hydrophobic polymers, using emulsion solvent evaporation, a single-step nanoprecipitation method, or microfluidic devices. Exemplary emulsion-based procedures are described below.

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

Methods for forming microspheres using solvent evaporation techniques are described in E. Mathiowitz et al., J. Scanning Microscopy, 4:329 (1990); L. R. Beck et al., Fertil. Steril., 31:545 (1979); L. R. Beck et al, Am. J Obstet. Gynecol., 135(3) (1979); S. Benita et al., J Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microparticles/nanoparticles. This method is useful for relatively stable polymers like polyesters and polystyrene.

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns.

Nanoparticles can be prepared using microfluidic devices. A polymeric material is mixed with a drug or drug combinations in a water miscible organic solvent. The water miscible organic solvent can be one or more of the following: acetone, ethanol, methanol, isopropyl alcohol, acetonitrile and Dimethyl sulfoxide (DMSO). The resulting mixture solution is then added to an aqueous solution to yield nanoparticle solution. The peptides or fluorophores or drugs may be associated with the surface of, encapsulated within, surrounded by, and/or distributed throughout the polymeric matrix of the particles.

In nanoprecipitation, the polymer and active agent (e.g., nucleic acids) are co-dissolved in a selected, water-miscible solvent, for example DMSO, acetone, ethanol, acetone, etc. In a preferred embodiment, active agent and polymer are dissolved in DMSO. The solvent containing the polymer and active agent is then drop-wise added to an excess volume of stirring aqueous phase containing a stabilizer (e.g., poloxamer, PLURONIC®, and other stabilizers known in the art). Particles are formed and precipitated during solvent evaporation. To reduce the loss of polymer, the viscosity of the aqueous phase can be increased by using a higher concentration of the stabilizer or other thickening agents such as glycerol and others known in the art. Lastly, the entire dispersed system is centrifuged, and the nucleic acid-loaded polymer nanoparticles are collected and optionally filtered. Nanoprecipitation-based techniques are discussed in, for example, U.S. Pat. No. 5,118,528.

Advantages to nanoprecipitation include: the method can significantly increase the encapsulation efficiency of drugs that are polar yet water-insoluble, compared to single or double emulsion methods (Alshamsan, Saudi Pharmaceutical Journal, 22(3):219-222 (2014)). No emulsification or high shear force step (e.g., sonication or high-speed homogenization) is involved in nanoprecipitation, therefore preserving the conformation of nucleic acids. Nanoprecipitation relies on the differences in the interfacial tension between the solvent and the nonsolvent, rather than shear stress, to produce nanoparticles. Hydrophobicity of the drug will retain it in the instantly-precipitating nanoparticles; the un-precipitated polymer due to equilibrium is “lost” and not in the precipitated nanoparticle form.

2. Liposomes, Micelles and Multilamellar Vesicles

In some embodiments, the nanoparticles are negatively charged liposomes, micelles, or multilamellar vesicles having diameters in the nanometer range. Liposomes are spherical vesicles composed of concentric phospholipid bilayers separated by aqueous compartments. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J., 124, 58P (1971)). Hydrophobic compounds associate with the lipid phase, while hydrophilic compounds associate with the aqueous phase.

Negatively charged liposomes and micelles can be prepared using anionic lipids, attaching negatively charged terminal moieties to anionic or cationic or neutral lipids or to a linker such as PEG. Controlling the zeta potential of liposomes by varying the lipid content, lipid ratio, level of cholesterol, level of negatively charged moieties, and the presence or absence of terminal PEG resides are well known in the art. See, for example, Nie, et al., Theranostics, 2(11):1092-1103 (2012), which describes that zeta potential has a close relationship with the charge on the cholesterol derivatives. Liposomes prepared from HEPC/Chol (70/30, mol/mol) were neutral (−2.0 mV). While adding 15 mol % lysine-based cholesterol with positive charge (CHLYS) or cholesterol hemisuccinate with negative charge (CHEMS), zeta potential changed to +37.2 mV or −33.5 mV, respectively (Table 1). Introduction of PEG to charged liposomes reduced the absolute value of surface charges (from +37.2 mV to +15.2 mV, or from −33.5 mV to −14.6 mV, respectively). No significant change was found in the conventional neutral liposomes.

Liposomes are formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In a preferred embodiment, the liposomes contain a phosphaditylcholine (PC) head group, and preferably sphingomyelin. In another embodiment, the liposomes contain DPPC. In a further embodiment, the liposomes contain a neutral lipid, preferably 1,2-dioleoylphosphatidylcholine (DOPC).

In certain embodiments, the liposomes are generated from a single type of phospholipid.

Cationic lipids include, but are not limited to, N-[1-(2,3-dioleoyloxy)propyl]-N,N,N-trimethyl ammonium salts, also referred to as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine), with cholesterol being most preferred.

B. Terminal Moieties

Nanoparticles can include one or more of the polymers or lipids having a terminal moiety. Terminal moieties include, but are not limited to, moieties that impart negative charge (i.e., negative charge moieties), targeting moieties, and detectable labels. Nanoparticles can have two or more terminal moieties. For example, in some embodiments, the particles are formed of a blend of particles having different terminal moieties.

Two methods to incorporate terminal moieties into the nanoparticles include: i) conjugation of terminal moiety to the hydrophilic region (e.g. PEG) of polymers or lipids prior to nanoparticle preparation; and ii) incorporation of terminal moieties onto nanoparticles after nanoparticle preparation.

1. Negatively Charged Moieties

The nanoparticles are negatively charged. Thus, some or all of the polymers forming the particle typically have a terminal moiety that imparts a negative charge to the particle. In some embodiments, the negatively charged moiety is a chemical modification to the polymer or lipid itself that imparts a negative charge to the polymer or lipid. In some embodiments, the negatively charge moiety is a separate, negatively-charged component that is conjugated to the polymer, lipid, etc.

The terminal moiety that imparts a negative charge to the nanoparticles can be an acidic group or an anionic group. Examples of acidic groups include, but are not limited to, carboxylic acids, protonated sulfates, protonated sulfonates, protonated phosphates, singly- or doubly protonated phosphonates, and singly- or doubly protonated hydroxamate. The corresponding salts of these acidic groups form anionic groups such as carboxylates, sulfates, sulfonates, singly- or doubly deprotonated phosphate, singly- or doubly deprotonated phosphonate, and hydroxamate.

2. Targeting Moieties

In some embodiments, some of the polymers of the nanoparticle include terminal moieties that enhance delivery of the particles to macrophage or another phagocytic cell type.

Exemplary targeting molecules include proteins, peptides, nucleic acids, lipids, saccharides, or polysaccharides, or small molecules that bind to one or more targets associated with or specific to macrophage. In particular embodiments the targeting moiety is a protein, peptide, antibody or aptamer. The degree of specificity with which the particles are targeted can be modulated through the selection of a targeting molecule with the appropriate affinity and specificity. For example, a targeting moiety can be a polypeptide, such as an antibody that specifically recognizes a macrophage marker that is present exclusively or in higher amounts on macrophage relative to other cell types. Targeting molecules can be covalently bound to particles using a variety of methods known in the art. In some embodiments, the targeting moieties are covalently associated with the polymer, for example, via a linker cleaved at the site of delivery.

The nanoparticles can contain one or more polymer conjugates containing end-to-end linkages between the polymer and a targeting element or a detectable label.

Targeting agents can increase uptake in targeted cells, decrease uptake in non-targeted cells, reduce toxicity to healthy cells, and combinations thereof.

The targeting element of the nanoparticle can be an antibody or antigen binding fragment thereof. The targeting elements should have an affinity for a cell-surface receptor or cell-surface antigen on the target cells and result in internalization of the particle within the target cell.

The targeting element can specifically recognize and bind to a target molecule specific for macrophage or another phagocytic cell. The target molecule can be a cell surface polypeptide, lipid, or glycolipid. The target molecule can be a receptor that is selectively expressed on a specific cell surface, a tissue or an organ. Exemplary cell surface markers expressed by macrophage include, but are not limited to, F4/80 and CD204, and macrophage-specific targeting is exemplified in the experiments below with anti-F4/80 and anti-CD204 antibodies.

The targeting peptides can be covalently associated with the polymer of the outer shell and the covalent association can be mediated by a linker.

The examples also show that targeting moieties are not necessary, and specific internalization of nanoparticles by macrophage can be enhanced by negative charge alone. Thus in some embodiments, the nanoparticles do not include a targeting moiety

C. Therapeutic, Prophylactic and Diagnostic Agents

The nanoparticles typically include a therapeutic, prophylactic and/or diagnostic agent. The active agent can be selected based on the intended treatment or therapy. Agents can be small molecules (less than 2000 D, more typically less than 1000 D), peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), lipids, saccharides or polysaccharides, or combinations thereof.

In one embodiment, nanoparticles are used to specifically deliver therapeutics to macrophages or other phagocytic cells, thereby modulating macrophage activity. Exemplary agents include, for example, therapeutic drugs (e.g., immunomodulatory drugs), invasive and non-invasive imaging agents, etc. Thus, the methods can be therapeutic or diagnostic in nature.

Solutions and dispersions of the active compounds as the free acid or base or pharmacologically acceptable salts thereof can be prepared in water or another solvent or dispersing medium mixed with one or more pharmaceutically acceptable excipients including, but not limited to, surfactants, dispersants, emulsifiers, pH modifying agents, viscosity modifying agents, and combination thereof.

1. Immunomodulators

Specific examples of immunomodulatory agents include, but are not limited to, interferon, anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steriodal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anti-PD1 antibodies, PD1 inhibitors, anti-B7-H1, anti-CTLA-4, CTLA-4-Ig anticholinergic agents (e.g., ipratropium bromide and oxitropium bromide), sulphasalazine, penicillamine, dapsone, and antihistamines.

In some embodiments, the active agent is a TGF-β pathway inhibitor. In some embodiment, the active agent is a TGF-β inhibitor that is trafficked by macrophage to a site of inflammation or degeneration where the inhibitor can renormalize overly activated TGF-β pathway. In another embodiment, the active agent is a TGF-β inhibitor that is delivered to peripheral macrophages and/or monocytes in a cell-specific manner. TGF-β inhibitors are known in the art and include, for example,

Lovastin;

SB-505124: a selective inhibitor of TGFβR for ALK4, ALK5 with IC50 of 129 nM and 47 nM, respectively.

SB-525334: a potent activin receptor-like kinase (ALK5)/type I TGFβ-receptor kinase inhibitor with IC50 of 14.3 nM.

LY364947: a selective small molecule inhibitor of the TGFβ type I receptor kinase with IC50 of 59 nM.

GW788388: a potent selective inhibitor of TGF-beta type I receptor and ALK5 with IC50 values of 0.093 and 0.018 μM.

LY2157299: an ALK5 kinase inhibitor with IC50 of 56 nM.

In certain embodiments, anti-TNF alpha antibodies (e.g., Remicade), CTLA-4-Ig (e.g., orencia), and/or TNFR-Ig (e.g., Enbrel) are used to prevent, treat, and/or manage an autoimmune disease, inflammatory disease, rheumatoid arthritis, and/or transplant rejection.

Other therapies which are known to be useful, or which has been used or is currently being used for the prevention, management, and/or treatment of a disease that is affected by immune function or response or inflammation can be used.

2. Other Active Agents

Other active include chemotherapeutic agents, anti-infectives including anti-viral agents (e.g., nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythomycin, penicillin, mithramycin, and anthramycin (AMC)), and agents that are active in the brain including dopamine and serotonin antagonists or agonists, can be delivered.

See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W. B. Saunders, Philadelphia, 1996, and Physicians' Desk Reference (61st ed. 2007) for information regarding therapies (e.g., prophylactic or therapeutic agents) which have been or are currently being used for preventing, treating and/or managing disease or disorder.

3. Imaging Agents

Non-limiting examples of imaging moieties that can be used in imaging agents include ¹¹C, ¹³N, ¹⁸F, ⁷⁶Br, ¹²³I, ¹²⁴I, ¹²⁵I, ¹³¹I, ⁹⁹mTc, ⁹⁵Tc, ¹¹¹In, ⁶²Cu, ⁶⁴Cu, ⁶⁷Ga, and ⁶⁸Ga. In some embodiments, the imaging moiety is selected from the group consisting of ¹⁸F, ⁷⁶Br, ¹²⁴I, ¹³¹I, ⁶⁴Cu, ⁸⁹Zr, ⁹⁹mTc, and ¹¹¹In. The imaging moiety can directly associated (i.e., through a covalent bond) with the nanocarrier, or can be part of another molecule that is incorporated onto or into the nanocarrier. In some embodiments, a composition including imaging agents or a plurality of imaging agents is enriched with an isotope such as a radioisotope, referred to as being “isotopically enriched.” An “isotopically enriched” composition refers to a composition including a percentage of one or more isotopes of an element that is more than the naturally occurring percentage of that isotope. For example, a composition that is isotopically enriched with a fluoride species may be “isotopically enriched” with fluorine-18 (¹⁸F). Thus, with regard to a plurality of compounds, when a particular atomic position is designated as ¹⁸F, it is to be understood that the abundance (or frequency) of ¹⁸F at that position (in the plurality) is greater than the natural abundance (or frequency) of ¹⁸F, which is essentially zero.

Other specific examples include, but are not limited to, superparamagnetic iron oxide (SPIO; an MRI tracing agent), Gadolinium (contrast agent that may be given during MRI scans; highlights areas of tumor or inflammation); PET and Nuclear Medicine Imaging Agents, such as 64Cu-ATSM (64Cu diacetyl-bis(N4-methylthiosemicarbazone), FDG (18F-fluorodeoxyglucose, radioactive sugar molecule, that, when used with PET imaging, produces images that show the metabolic activity of tissues); 18F-fluoride (imaging agent for PET imaging of new bone formation); FLT (3′-deoxy-3′-[18F]fluorothymidine, radiolabeled imaging agent that is being investigated in PET imaging for its ability to detect growth in a primary tumor); FMISO (18F-fluoromisonidazole, imaging agent used with PET imaging that can identify hypoxia (low oxygen) in tissues); 18F-FDDNP or 11C-PIB (PET-based radioisotope-conjugated imaging agents for detection of misfolded Aβ and misfolded tau proteins); Gallium (attaches to areas of inflammation, such as infection and also attaches to areas of rapid cell division, such as cancer cells); Technetium-99m (radiolabel many different common radiopharmaceuticals; used most often in bone and heart scans);Thallium (radioactive tracer typically used to examine heart blood flow); and combinations thereof.

D. Pharmaceutical Compositions

The particles can be formulated with appropriate pharmaceutically acceptable carriers into pharmaceutical compositions for administration to an individual in need thereof. The formulations can be administered parenterally (e.g., by injection or infusion). It may be possible to administer orally, nasally, via a mucosal surface such as the buccal, vaginal, rectal or lung mucosa.

In some embodiments, the pharmaceutical composition includes one or more cations such as Ca²⁺ or Mg²⁺. A.s illustrated in the experiments below, cations can enhance systemic circulation and subsequent uptake of negatively charged particles by macrophage.

The particles can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

Enteral formulations are prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins.

Carrier also includes all components of the coating composition, which may include plasticizers, pigments, colorants, stabilizing agents, and glidants. Formulations can be prepared using one or more pharmaceutically acceptable excipients, including diluents, preservatives, binders, lubricants, disintegrators, swelling agents, fillers, stabilizers, and combinations thereof.

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Controlled release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

### III. Methods of Use

It has been discovered negatively charged particles exhibit (1) increased circulation and/or reduced clearance from circulation, (2) increased internalization by phagocytic cells such as macrophage, and (3) increase exocytosis from macrophage relative to other particles such as those that are positively charged or charge-neutral. These properties allow the cells to first efficiently take up nanoparticles encapsulating an active agent, and later discharge the nanoparticle at a distal location. The discharged cells can be internalized by target cells in the distal location thus delivering the active agent to the interior of the target cells; degrade following exocytosis, thus releasing the active agent into the microenvironment of the distal location; or a combination thereof. Macrophages that have taken up such nanoparticles are similar to ‘Trojan Horses” in the sense that they can deliver nanoparticles to sites to which macrophage are normally trafficked within the body, while avoiding degradation and other pathways that may reduce their bioavailability. For example, trafficking the nanoparticles within macrophage can allow the particles and their encapsulated active agent to avoid metabolism and off-target toxicity while also avoid physical impediments to drug delivery such as the blood-brain barrier.

In some embodiments, the negatively charged nanoparticles encapsulating the active agent are administered to a subject in need thereof, internalized by macrophage and/or other phagocytic cells, and trafficked to a site of inflammation or infection. The nanoparticles are released by the cell, which subsequently release the active agent. The active agent, now present in the inflammatory microenvironment, can act on cells or other biological materials in the microenvironment.

In some embodiments, the negatively charged nanoparticles encapsulating the active agent are administered to a subject in need thereof, internalized by macrophage and/or other phagocytic cells, and thereby modulating the activity state of the macrophage and/or other phagocytic cells.

In some embodiments, the active agents serve an autocrine-like and/or paracrine-like function. Thus, the active agent can act on the macrophage that delivered the nanoparticles to the microenvironment. The active agent can also act on other cells, including, but not limited to, other macrophages in the microenvironment.

The examples show that encapsulation of TGF-β1 antagonists leads to suppression of TGF-β1 in macrophages/monocytes in the brain and subsequent dissolution/clearance of Amyloid β aggregates, including plaques, reversal of synaptic damage and finally subsequent resolution of Alzheimer's disease. However, the same principle can be applied to encapsulation and release of a range of different therapeutic agents to treat a range of diseases and disorders both in the brain and other sites throughout the body.

Typically, an effective amount of negatively charged nanoparticle-encapsulated active agent can be used to treat a disease or disorder. The amount is typically effective to have a therapeutic effect at a site of disease or disorder in the subject when internalized by macrophage or other phagocytic cells.

Macrophages are “first responders” in many disease states. They are the primary immune cells that respond to an infection, wound, or any bodily injury. As such, macrophages alert the immune system (both innate and adaptive) to the nature of the dysfunction. Macrophages can be used to deliver immunomodulatory agents to a site of infection, inflammation, or tumor growth. Macrophages can also be stimulated to clear protein aggregates and/or cellular debris generated under pathophysiological conditions. Thus, the compositions can be used to treat cancer, inflammatory disorders, autoimmune diseases, neurodegenerative diseases. The compositions and methods be used to deliver active agents to sites of particulate matter including, but not limited to, bacteria, viruses, parasites, cell debris and even misfolded proteins including but not limited to Aβ or tau or α-synuclein in the brain.

Several neurodegenerative disorders have been associated with the presence of misfolded protein aggregates (“Protein Misfolding Disorders”). Protein Misfolding Disorders may include amyloidosis such as Alzheimer's disease (AD) or systemic amyloidosis; synucleinopathies such as Parkinson's disease (PD), Lewy body dementia (LBD); multiple system atrophy; and synuclein-related neuroaxonal dystrophy; type 2 diabetes; triplet repeat disorders such as Huntington's disease (HD); amyotrophic lateral sclerosis (ALS); transmissible spongiform encephalopathies (TSE) such as Creutzfeldt-Jakob disease and its variant (CJD, vCJD), kuru, Gerstmann-Straussler-Scheiker disease (GSS), and fatal familial insomnia (FFI), and the like. Misfolded aggregates of different proteins may be formed and accumulate. The misfolded protein aggregates might include Abeta, alpha-synuclein, tau, PrP. The misfolded aggregates may induce cellular dysfunction and tissue damage, among other effects.

As used herein, “Aβ” or “amyloid beta” or “beta amyloid” refers to a peptide formed via sequential cleavage of the amyloid precursor protein (APP). Various Aβ isoforms may include 38-43 amino acid residues. The Aβ protein may be formed when APP is sequentially processed, first by β- and then by γ-secretases. The Aβ may be a constituent of amyloid plaques in brains of individuals suffering from or suspected of having AD. Various Aβ isoforms may include and are not limited to Aβ40 and Aβ42. Various Aβ peptides may be associated with neuronal damage associated with AD.

As used herein, “tau” refers to proteins are the product of alternative splicing from a single gene, e.g., MAPT (microtubule-associated protein tau) in humans. Tau proteins include full-length and truncated forms of any of tau's isoforms. Various isoforms include, but are not limited to, the six tau isoforms known to exist in human brain tissue, which correspond to alternative splicing in exons 2, 3, and 10 of the tau gene. Three isoforms have three binding domains and the other three have four binding domains. Misfolded tau may be present in brains of individuals suffering from AD or suspected of having AD, or other tauopathies.

As used herein, “αS” or “alpha-synuclein” refers to full-length, 140 amino acid α-synuclein protein, e.g., “αS-140.” Other isoforms or fragments may include “αS-126,” alpha-synuclein-126, which lacks residues 41-54, e.g., due to loss of exon 3; and “αS-112” alpha-synuclein-112, which lacks residue 103-130, e.g., due to loss of exon 5. The αS may be present in brains of individuals suffering from PD or suspected of having PD. Various αS isoforms may include, and are not limited to, αS-140, αS-126, and αS-112. Various αS peptides may be associated with neuronal damage associated with PD.

Peripheral macrophages are potent phagocytes capable of clearing cellular debris and misfolded protein aggregates. Upon activation, macrophages and other peripheral monocytes can migrate to target tissues to eliminate cellular debris or protein aggregates. In one embodiment, an active agent is specifically delivered to peripheral macrophages by treatment of a subject in the need thereof with an active agent encapsulated in negatively charged nanoparticles, and the active agent released within that macrophage, thereby modulating the activity of the macrophage or other phagocytic cell. In one embodiment, a subject having a Protein Misfolding Disorder is treated with an active agent encapsulated in a negatively charged nanoparticle, leading to cell-specific activation of macrophages and clearance of misfolded protein aggregates. In one specific embodiment, the active agent results in migration of peripheral macrophages to the CNS. In one specific embodiment, the active agent is a TGF-β pathway inhibitor. In one embodiment, the active agent is combined with an imaging agent to allow tracking of targeted cells.

In some embodiments, the negatively charged nanoparticles encapsulating the active agent are administered to a subject in need thereof, internalized by macrophage and/or other phagocytic cells, and the encapsulated agent released within the macrophage or other phagocytic cell, thereby modulating the function, activity or other parameter of the macrophage. Macrophages can also be activated to directly clear protein aggregates, especially misfolded protein aggregates. In particularly preferred embodiments, the active agent is a TGF-β signaling inhibitor delivered to a macrophage or a macrophage-like cell in order to change the cell's phenotype in order to promote phagocytosis or clearance of misfolded protein(s). For example, experiments indicate that encapsulation of TGF-β1 antagonists lead to suppression of TGF-62 1 in peripheral macrophages, and subsequent clearance of Amyloid β aggregates, including plaques, reversal of synaptic damage and finally subsequent mitigation of Alzheimer's disease. Thus, blocking innate immune TGF-β signaling leads to desirable brain infiltration of peripheral macrophages and beneficial clearance of misfolded proteins such as Aβ, tau, or α-synuclein. However, the same principle can be applied to encapsulation and release of a range of different therapeutic agents to treat a range of diseases and disorders both in the brain and other sites throughout the body. As introduced above, this therapy is particularly effective for treating one or more symptoms of a disease involving cerebral amyloid aggregate in neurodegenerative diseases such as Alzheimer's disease, but also has application in other Protein Misfolding Disorders or disorders that exhibit a build-up of misfolded proteins, including, but not limited to, Parkinson's disease, amyotrophic lateral sclerosis, frontotemporal dementia, Lewy body dementia, traumatic brain injury Type II diabetes, atherosclerosis and other manifestations of cardiovascular disease, and autoimmune disease.

In some embodiments, the nanoparticles are utilized in methods of imaging. Imaging agents be incorporated in, or attached to, the polymers or lipids. The methods can include administering nanoparticles including an imaging agent to a subject, and imaging a region of the subject that is of interest. In some embodiments, a method of imaging includes (a) administering to a subject a nanocarrier that includes an imaging agent, and (b) acquiring at least one image of at least a portion of the subject. Suitable systems for imaging include, but are not limited to, magnetic resonance imaging (MRI), positron emission tomography (PET), single-photon emission computed tomography (SPECT) and optical imaging (OI).

In some embodiments, positron emission tomography (PET) is utilized for visualizing the distribution of the imaging agent within at least a portion of the subject. Imaging may include full body imaging of a subject, or imaging of a specific body region or tissue of the subject that is of interest (e.g., the brain). In some embodiments, a method may include diagnosing or assisting in diagnosing a disease or condition, assessing efficacy of treatment of a disease or condition, or imaging in a subject with a known or suspected disease or condition of the brain or central nervous system.

In some embodiment, an active agent is combined with an imaging agent in the composition.

As discussed above, the compositions can be used to prevent, reduce, delay, or inhibit the formation or aggregation of misfolded proteins; prevent, reduce, delay, or inhibit the level, formation, or production of amyloid proteins, such as amyloid beta, in a subject over time. The compositions are particularly useful for treating a subject with, or likely to develop, a proteinopathy, amyloidosis, or a tauopathy.

A. Proteinopathies

Compositions and methods of treating or preventing proteinopathies and amyloidosis are disclosed. For example, the methods can include administering to a subject in need thereof an effective amount of one or more of the compositions to reduce, delay, or inhibit the expression or accumulation of one or more misfolded proteins.

The compositions can be administrated to a subject in an effective amount to treat a proteinopathy, or symptom, characteristic or comorbidity thereof. Proteinopathies include, but are not limited to, Alzheimer's disease, cerebral β-amyloid angiopathy, Retinal ganglion cell degeneration in glaucoma, Prion diseases, Parkinson's disease and other synucleinopathies, Tauopathies, Frontotemporal lobar degeneration (FTLD), FTLD-FUS, Amyotrophic lateral sclerosis (ALS), Huntington's disease and other triplet repeat disorders, Familial British dementia, Familial Danish dementia, Hereditary cerebral hemorrhage with amyloidosis (Icelandic) (HCHWA-I), CADASIL, Alexander disease, Seipinopathies, Familial amyloidotic neuropathy, Senile systemic amyloidosis, Serpinopathies, AL (light chain) amyloidosis (primary systemic amyloidosis), AH (heavy chain) amyloidosis, AA (secondary) amyloidosis, Type II diabetes, Aortic medial amyloidosis, ApoAI amyloidosis, ApoAII amyloidosis, ApoAIV amyloidosis, Familial amyloidosis of the Finnish type (FAF), Lysozyme amyloidosis, Fibrinogen amyloidosis, Dialysis amyloidosis, Inclusion body myositis/myopathy, Cataract, Retinitis pigmentosa with rhodopsin mutations, Medullary thyroid carcinoma, Cardiac atrial amyloidosis, Pituitary prolactinoma, Hereditary lattice corneal dystrophy, Cutaneous lichen amyloidosis, Mallory bodies, Corneal lactoferrin amyloidosis, Pulmonary alveolar proteinosis, Odontogenic (Pindborg) tumor amyloid, Seminal vesicle amyloid, Cystic Fibrosis, Sickle cell disease, Critical illness myopathy (CIM).

In certain embodiments the subject has a mutation in a gene, such as the Aβ, ABri, ADan, superoxide dismutase, α-synuclein, huntingtin, ataxins, or neuroserpin genes, which could lead to accumulation of malformed protein or protein aggregates which could trigger a pathological cascade leading to clinical manifestation of a proteinopathy. The subject may or may not be exhibiting physical symptoms of the proteinopathy at the time treatment is initiated.

B. Amyloidosis

The compositions can also be administrated to a subject in an effective amount to treat amyloidosis, or symptom, characteristic or comorbidity thereof. In some embodiments, the amyloidosis is caused by the amyloid protein beta amyloid (Aβ), the amylin protein, the islet amyloid polypeptide, medin (AMed), Apolipoprotein AI (AApoA1), atrial natriuretic factor (AANF), Cystatin (ACys), IAPP (Amylin) (AIAPP), beta 2 microglobulin (Aβ2M), Transthyretin (ATTR), Gelsolin (AGel), Lysozyme (ALys), huntingtin, keratoepithelin (Aker), calcitonin (ACal), alpha-synuclein, Prolactin (APro), serum amyloid A, (AA), S-IBM, immunoglobulin light chain AL (AL), or PrPSc (APrP).

Amyloidosis includes, but is not limited to, diseases such as Alzheimer's disease (beta amyloid), aortic medial amyloid (Medin), atherosclerosis (Apolipoprotein AI), cardiac arrhythmias and isolated atrial amyloidosis (atrial natriuretic factor), cerebral amyloid angiopathy (beta amyloid), cerebral amyloid angiopathy—Icelandic type (Cystatin), diabetes mellitus type 2 (IAPP-Amylin), dialysis related amyloidosis (beta 2 microglobulin), familial amyloid polyneuropathy, (transthyretin), Finnish amyloidosis (gelsolin), hereditary non-neuropathic systemic amyloidosis (lysozyme), Huntington's disease, (Huntingtin), lattice corneal dystrophy (keratoepithelin), medullary carcinoma of the thyroid (calcitonin), multiple myeloma (paraprotein), Parkinson's disease (alpha-synuclein) prolactinomas (prolactin), rheumatoid arthritis (serum amyloid A), Sporadic Inclusion Body Myositis (S-IBM); systemic AL amyloidosis (immunoglobulin light chain AL), primary cutaneous amyloidosis, AA amyloidosis, senile amyloid of atria of heart, familial visceral amyloidosis, Cerebral amyloid angiopathy (British-type and Danish-type), medullary carcinoma of the thyroid, familial corneal amyloidosis, prion disease systemic amyloidosis, leptomeningeal amyloidosis, haemodialysis-associated amyloidosis, and transmissible spongiform encephalopathies (PrPSc).

Examples of transmissible spongiform encephalopathies include, but are not limited to, human diseases such as Creutzfeld Jakob Disease, variant Creutzfeld Jakob Disease, Gerstmann-Sträussler-Scheinker syndrome (GSS), fatal familial insomnia (FFI), kuru and Alpers' syndrome, and non-human diseases such as bovine spongiform encephalopathy (B SE, commonly known as mad cow disease) in cattle, chronic wasting disease (CWD) in elk and deer, and scrapie in sheep, transmissible mink encephalopathy, feline spongiform encephalopathy and ungulate spongiform encephalopathy.

C. Abeta-Related Diseases

Additional embodiments provide compositions and methods to treat a disease characterized by increased amyloid beta expression, deposition, aggregation or plaque formation. For example, a method of treating a disease or disorder characterized by increased amyloid beta expression, deposition, aggregation or plaque formation can include administering to a subject in need thereof a composition including an effective amount of a composition to reduce, delay, or inhibit the level, formation, or production of amyloid beta in the subject compared to a control.

Abeta-related diseases and disorders include, but are not limited to, Alzheimer's disease, cerebral amyloid angiopathy (also known as congophilic angiopathy), Lewy body dementia, retinal ganglion cell degeneration (such as in glaucoma), sporadic inclusion body myositis (sIBM) and hereditary inclusion body myopathy (hIBM).

The Abeta-related disease or diseases treated using the method is not Alzheimer's disease or Lewy body dementia.

Abeta is formed after sequential cleavage of the amyloid precursor protein (APP), a transmembrane glycoprotein of undetermined function. APP can be processed by α-, β- and γ-secretases; Abeta protein is generated by successive action of the β and γ secretases. The γ secretase, which produces the C-terminal end of the Abeta peptide, cleaves within the transmembrane region of APP and can generate a number of isoforms of between 36 and 43 amino acid residues in length. The most common isoforms are Abeta40 and Abeta42; the longer form is typically produced by cleavage that occurs in the endoplasmic reticulum, while the shorter form is produced by cleavage in the trans-Golgi network. The Abeta40 form is the more common of the two, but Abeta42 is the more fibrillogenic and is thus associated with disease states.

In some embodiments, the compositions are used to reduce, prevent or delay the amyloid beta expression, deposition, aggregation or plaque formation in a subject in need thereof. The method typically includes administering to the subject a composition including an effective amount of a composition to reduce, delay, or inhibit the level, formation, or production of amyloid beta in the subject compared to a control. The method can include treating subjects that have not yet been diagnosed with a specific disease or disorder.

D. Tauopathies

The compositions and methods can also be used to treat diseases characterized by increased tau expression or pathologies associated with the aggregation of tau protein in the brain. For example, a method of treating a disease or disorder characterized by increased tau expression, tau aggregation, or pathologies associated with the aggregation of tau protein in the brain can include administering to a subject in need thereof a composition including an effective amount of a composition to reduce, delay, or inhibit tau expression or aggregation in the subject compared to a control.

Examples of tauopathies and conditions associated therewith include, but are not limited to Alzheimer's disease, Argyrophilic grain disease (AGD), Chronic Traumatic Encephalopathy (CTE), Dementia pugilistica (chronic traumatic encephalopathy), frontotemporal dementia, frontotemporal lobar degeneration, gangliocytoma, Ganglioglioma, gangliocytoma, Lytico-Bodig disease (Parkinson-dementia complex of Guam), meningioangiomatosis, Frontotemporal dementia and Parkinsonism linked to chromosome 17 (FTDP-17), Pick's disease, Progressive supranuclear palsy, subacute sclerosing panencephalitis,tangle-predominant dementia, lead encephalopathy, tuberous sclerosis, Hallervorden-Spatz disease, lipofuscinosis, corticobasal degeneration.

In some embodiments the taupathy is a non-Alzheimer's tauopathy. Non-Alzheimer's tauopathies are sometimes grouped together as “Pick's complex” or “Pick's dementias.”

The method can include treating subjects that have not yet been diagnosed with a specific disease or disorder.

E. Neurodegenerative Diseases

The compositions and methods herein can be used to treat subjects with a neurodegenerative disease or disorder. Exemplary neurodegenerative diseases include, but are not limited to, Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies, Lacunar syndromes, Hydrocephalus, Wernicke-Korsakoff's syndrome, post-encephalitic dementia, cancer and chemotherapy-associated cognitive impairment and dementia, and depression-induced dementia and pseudodementia.

F. Cancers

The compositions and methods can be used to treat cancer. The types of cancer that can be treated with the provided compositions and methods include, but are not limited to, cancers such as vascular cancer such as multiple myeloma, adenocarcinomas and sarcomas, of bone, bladder, brain, breast, cervical, colo-rectal, esophageal, kidney, liver, lung, nasopharangeal, pancreatic, prostate, skin, stomach, and uterine. In some embodiments, the compositions are used to treat multiple cancer types concurrently. The compositions can also be used to treat metastases or tumors at multiple locations.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The examples below indicate that the viruses and methods herein are useful for treating cancer, particular brain tumors, in vivo.

The methods are particularly useful in treating brain tumors. Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells, lymphatic tissue, blood vessels), in the cranial nerves, in the brain envelopes (meninges), skull, pituitary and pineal gland, or spread from cancers primarily located in other organs (metastatic tumors). Examples of brain tumors include, but are not limited to, oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma. In a particular embodiment, the cancer is glioblastoma multiforme. In another embodiment, the cancer is medulloblastoma.

“Primary” brain tumors originate in the brain and “secondary” (metastatic) brain tumors originate from cancer cells that have migrated from other parts of the body. Primary brain cancer rarely spreads beyond the central nervous system, and death results from uncontrolled tumor growth within the limited space of the skull. Metastatic brain cancer indicates advanced disease and has a poor prognosis. Primary brain tumors can be cancerous or noncancerous. Both types take up space in the brain and may cause serious symptoms (e.g., vision or hearing loss) and complications (e.g., stroke). All cancerous brain tumors are life threatening (malignant) because they have an aggressive and invasive nature. A noncancerous primary brain tumor is life threatening when it compromises vital structures (e.g., an artery). In a particular embodiment, the compositions and methods are used to treat cancer cells or tumors that have metastasized from outside the brain (e.g., lung, breast, melanoma) and migrated into the brain.

The compositions and methods can be used to treat brain cancer, cancer that can metastasize to the brains, for example lung cancer, breast cancer, and skin cancer such as melanoma.

The present invention will be further understood by reference to the following non-limiting examples.

### EXAMPLES

### Example 1

### Negatively Charged Nanoparticles are More Efficiently Taken Up by Macrophages Through Cation Mediated Complexation

Materials Methods

Nanoparticles (NP):

(Size˜100-300 nm) terminated with the following:

PLGA-PEG-OMe: 5000 da-2000 da (methoxy terminated)-→Neutral

PLGA-PEG-COOH: 5000 da-2000 da (carboxy terminated)-→Negative charge

PLGA-PEG-NH2: 5000 da-2000 da (amine terminated)->Positive charge

Encapsulated Fluorescent Indicators Reporting on Particle Intracellular Trafficking:

Coumarin 6 or Rhodamine were encapsulated in NPs. Coumarin-6 does not leach out easily from particle and indeed reports on particle trafficking due to its hydrophobic equivalency to the PLGA matrix. C6, therefore, avidly associates with the polymer matrix, even during degradation.

Particle-Macrophage Incubation Medium Containing: 1× of Mg²⁺: Same concentration of DMEM media=0.09 mg/mL of MgSO₄ 1× of Ca²⁺: Same concentration of DMEM media=0.2 mg/mL of CaCl₂

Internalization Experiment:

Macrophage cell line (RAW 264.7) 10⁵ cells/well, 24 hr incubation with Coumarin 6 (500 mg/mL) encapsulated nanoparticle

Mg2+ 1× is same concentration as DMEM media (0.09 mg/mL).

Cells imaged use a Carestream Multispectral imaging pro.

Chelation Experiment:

EDTA: same concentration as Mg ion

24 hr treatment

Particles 250 μg/mL

Results

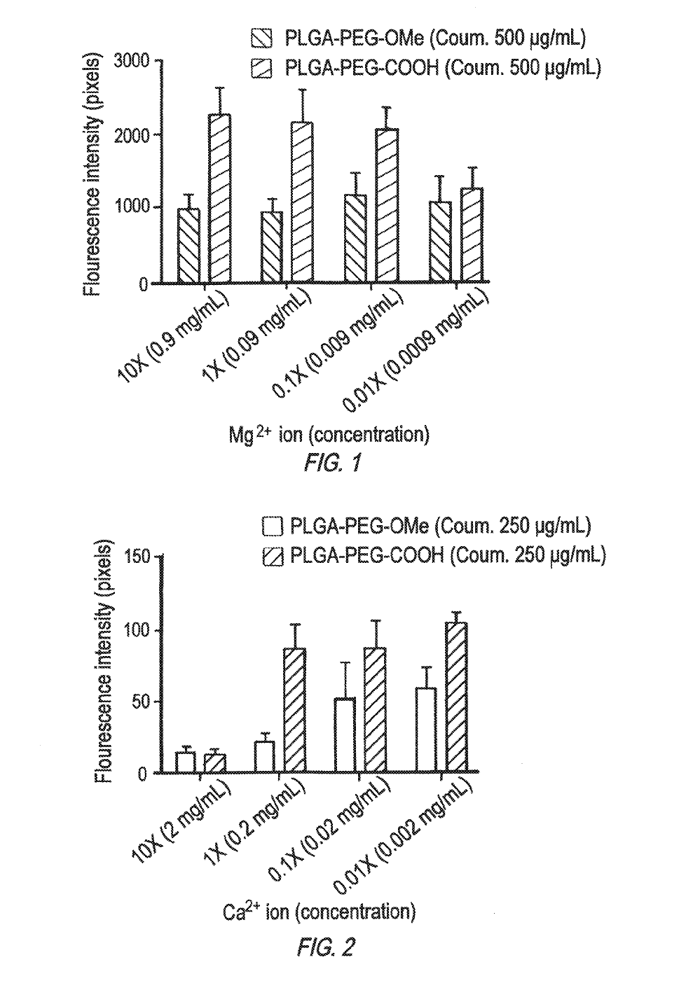

Experiments were designed to determine the impact of particle surface, particle charge, ionic species, and time on the internalization of particles by macrophage. The results of the experiments are illustrated in FIGS. 1-6, which collectively reveal that negatively charged nanoparticles are more efficiently taken up by macrophages through cation-mediated complexation prior to macrophage uptake.

Macrophages are negatively charged. It is counterintuitive that a negatively charged nanoparticle would be preferentially taken up versus a neutral or positively charged counterpart, because the conventional wisdom holds that opposite charges attract whereas similar charges repel.

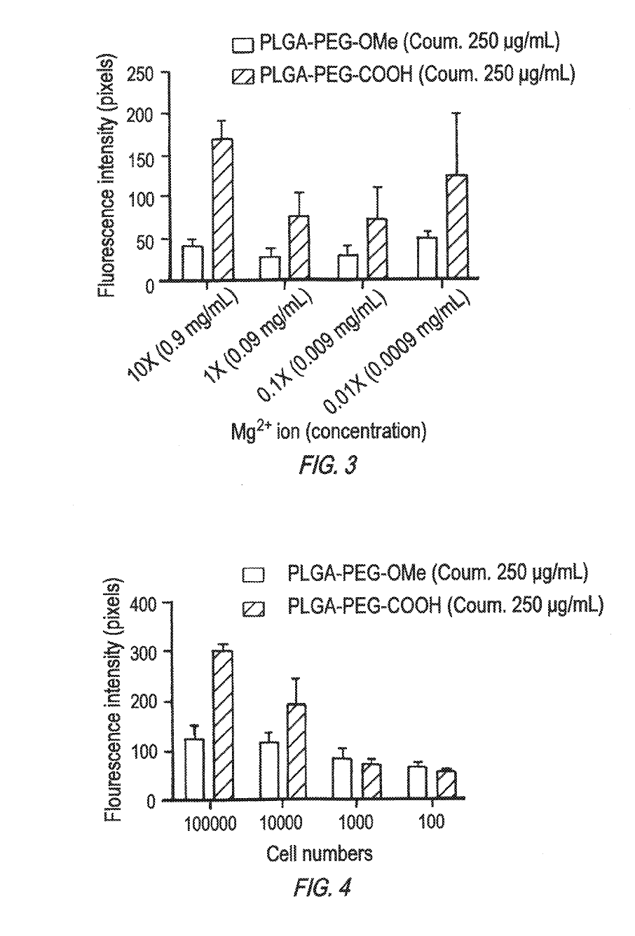

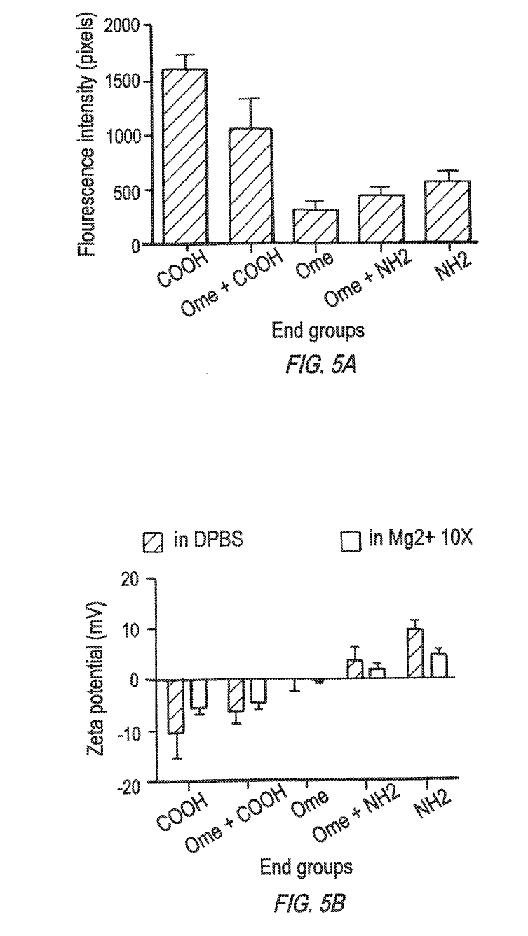

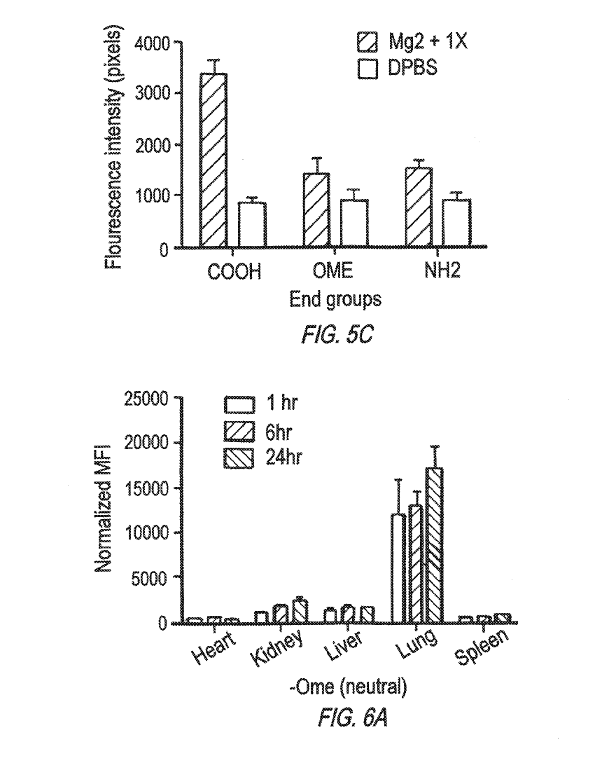

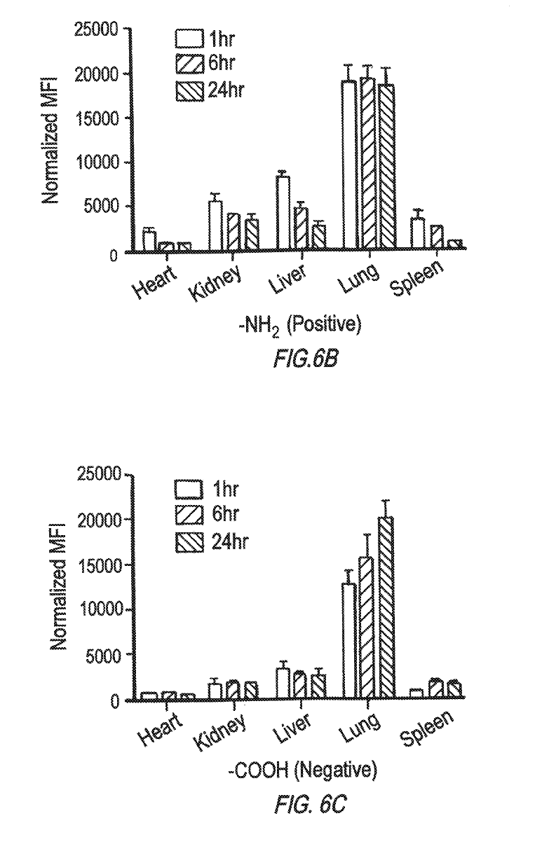

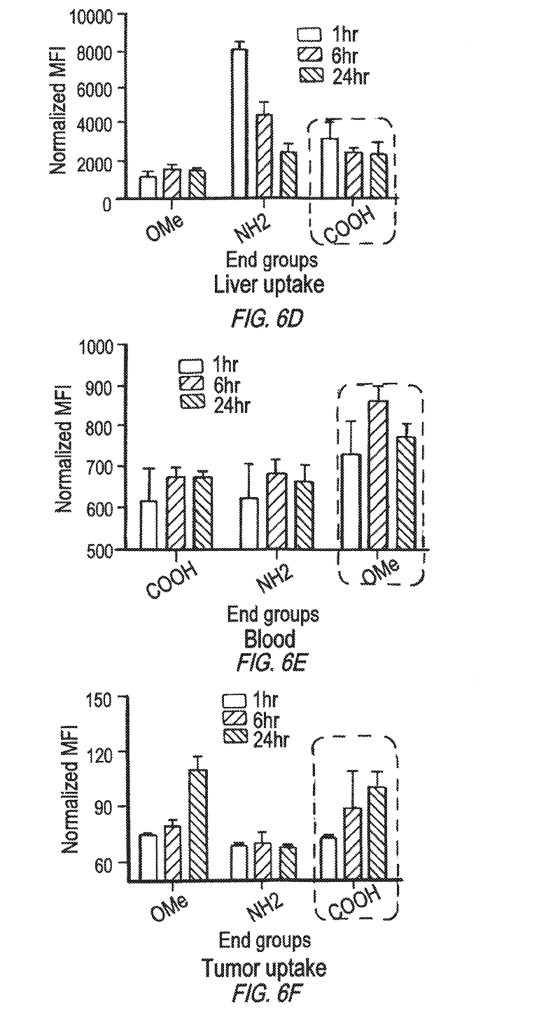

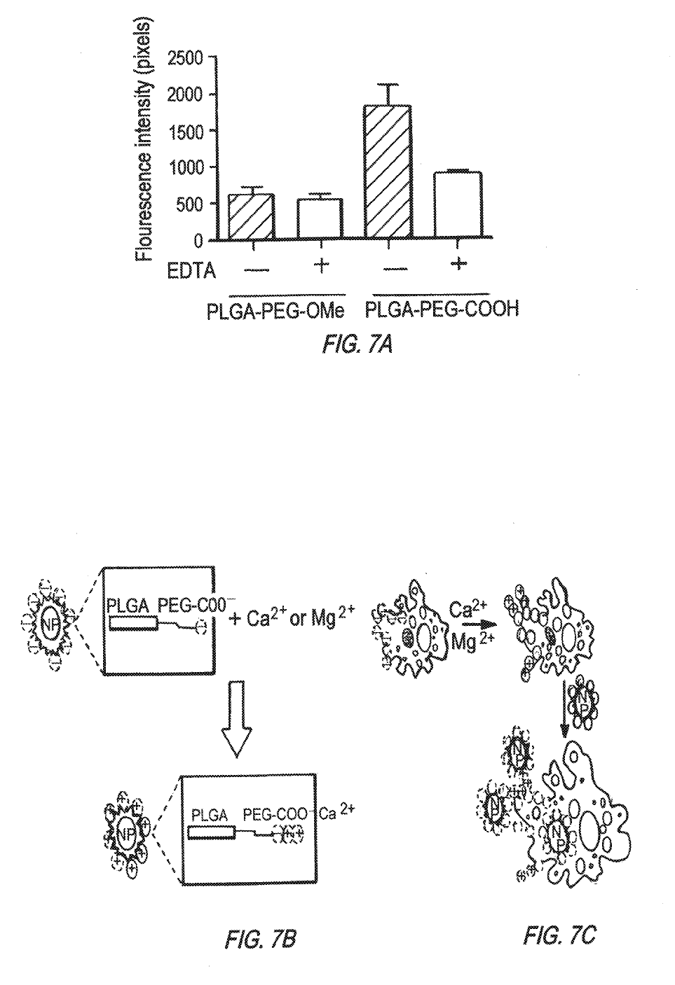

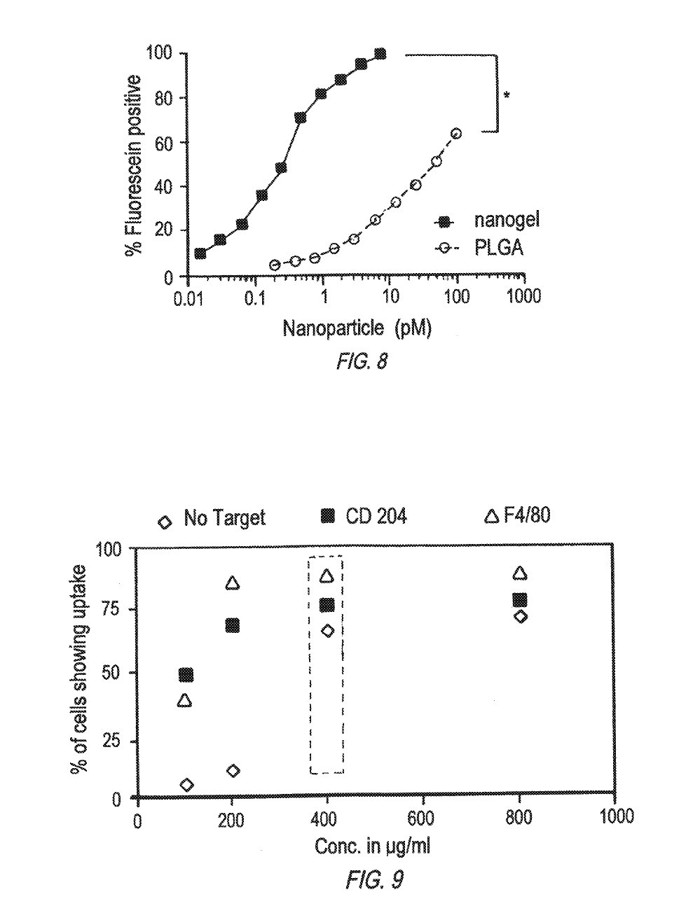

FIG. 1 is a bar graph showing the effect of magnesium (Mg²⁺) ion concentration on macrophage internalization of Coumarin 6-loaded (500 μg/ml) (Fluorescence intensity (pixels)) PLGA-PEG-OMe and PLGA-PEG-COOH nanoparticles (24 hours). FIG. 2 is a bar graph showing the effect of calcium (Ca²⁺) ion concentration on macrophage internalization of Coumarin 6-loaded (250 μg/ml) (Fluorescence intensity (pixels)) PLGA-PEG-OMe and PLGA-PEG-COOH nanoparticles (48 hours). FIG. 3 is a bar graph showing the effect of magnesium (Mg²⁺ ) ion concentration on macrophage internalization of Coumarin 6-loaded (250 μg/ml) (Fluorescence intensity (pixels)) PLGA-PEG-OMe and PLGA-PEG-COOH nanoparticles (48 hours). FIG. 4 is a bar graph showing the effect of cell number on macrophage internalization of Coumarin 6-loaded (250 μg/ml) (Fluorescence intensity (pixels)) PLGA-PEG-OMe and PLGA-PEG-COOH nanoparticles. FIGS. 5A-5B are bar graphs showing the effect of nanoparticle end group on macrophage uptake (5A) and nanoparticle charge (5B). FIG. 5C is a bar graph showing effect of the combination of nanoparticle end group and magnesium (Mg2+) concentration on macrophage uptake. FIGS. 6A-6C are bar graphs showing the impact of nanoparticle charge (-Ome (neutral) in 6A, —NH2 (positive) in 6B, and —COOH (negative) in 6C) on biodistribution to the heart, kidney, liver, lung, and spleen at 1, 6, and 24 hours. FIGS. 6D-6F are bar graphs showing the impact of nanoparticle end group (-Ome (neutral), —NH2 (positive), and —COOH (negative)) on uptake in liver (6D), blood (6E), and tumor (6F). FIG. 7A is a bar graph showing the effect of negative charge chelating EDTA on macrophage internalization of PLGA-PEG-Ome and PLGA-PEG-COOH nanoparticles. FIG. 7B is an illustration the proposed effect of cations on nanoparticles with negatively charged end groups. FIG. 7C is an illustration of a proposed mechanism of negatively charged nanoparticle internalization by negatively charged cells such as macrophage.

In vivo biodistribution is illustrated in FIGS. 6A-6F and shows that particles with neutral charge enhance circulation, exhibit minimal uptake in macrophage, and localization in abnormal vasculature (tumors); particles with positive charge are retained in organs (ex: liver), but are not efficiently taken up by macrophage; and particles with negative charge show prolonged circulation, are efficiently taken up by macrophage, and exhibit enhanced circulation (via a macrophage mediated carrier mechanism).

These results show negatively charged particles can be used to deliver cargo to macrophage, which can then be used as “Trojan Horses” to deliver cargo to other sites within the body to which macrophage traffic, for example, to Alzheimer's disease plaques. In another embodiment, the nanoparticles alter the activation state or activity or phenotype of the macrophages that are carrying them in a cell autonomous or non-autonomous fashion.

Such a delivery strategy can be preferred, for example, when there is a need for longer circulation times for cell encounters and/or to increase efficient uptake by cells that encountered the NPs.

Mechanisms of Action:

Because Macrophages are negatively charged they would be expected to repel negatively charged NP. However, the experiments described above reveal that negative charge is modulated by cations (Mg2+ and Ca2+) that are present in vivo. Positively charged NP are not taken up as efficiently. It may be that the NPs need to circulate in free form to reach macrophages for efficient uptake. Positively charged NP are non-discriminately taken up by various cells and organs. Negatively charged NP are initially repelled or slightly positively modulated by cationic species and then efficiently taken up by macrophage.

To validate this mechanism, an internalization experiment was carried out in the presence of EDTA. The addition of EDTA removes the negative charge through chelation. The charge-mediated internalization of NP by macrophage is reduced. However, EDTA has no impact on internalization of neutral particles. See FIG. 7A.

Thus, efficient uptake by macrophage is a balance between time in circulation and time for uptake. Positively charged NP: too little time in circulation=fast clearance=inefficient macrophage uptake. Neutral NP: long circulation time=better macrophage encounter but surface charge not sufficiently modulated for uptake by macrophage. The negative charge provides the relatively longer circulation time, and in the presence of cations in the body such as Mg2+ and Ca2+, the negative charge is modulated and macrophages efficiently take up the neutralized NP. See FIGS. 7B-7C.

### Example 2

### Particle Elasticity and Composition Differentially Impact Uptake

Materials and Methods

Liposomal NP and PLGA NPs are the same size (100-150 nm) and loaded with the same level fluorescein for analysis.

Results

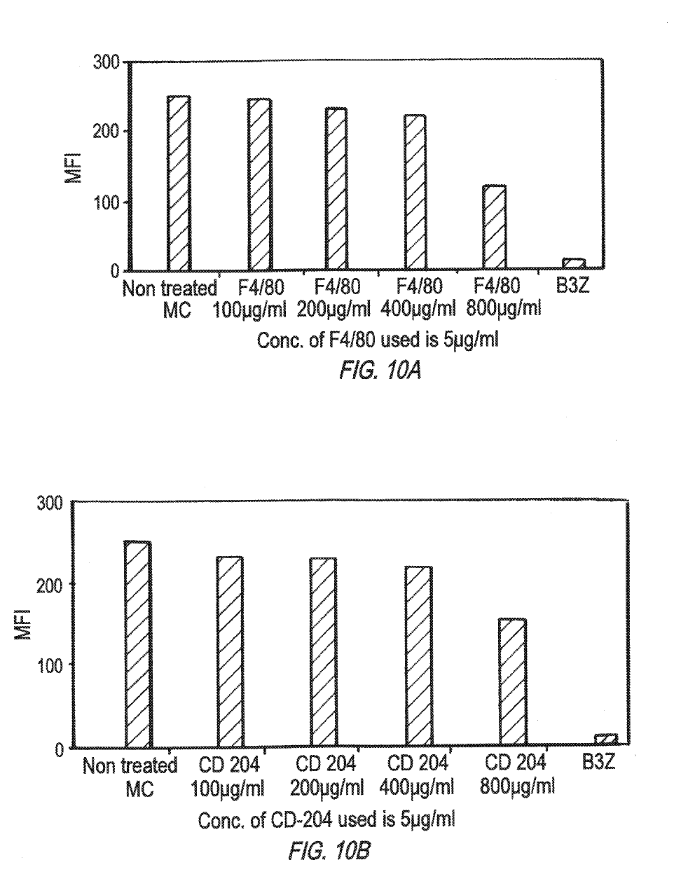

Enhanced uptake of lipidated nanoparticles (softer) was compared to PLGA (lower elasticity) in macrophages and other phagocytic cells. Liposomal particles are generally, “softer” on the surface and “squishier” compared to PLGA nanoparticles. The results are shown in FIG. 8. FIG. 8 is a bar graph comparing macrophage internalization of PLGA nanoparticles and lapidated nanoparticles.

### Example 3

### Targeting Can Reduce Dosage

Materials and Methods

Anti-F480 and anti-CD204 were used to exemplify the impact of addition of targeting moieties on the internalization of NP in macrophages. Incubation time=2 hours.

Results

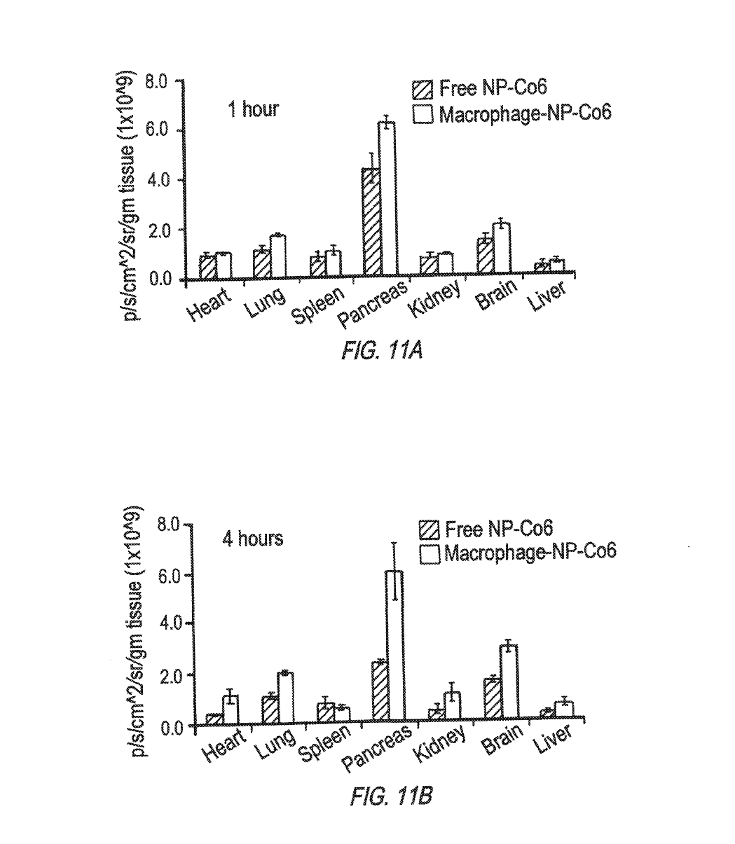

An assay was designed to test the impact of macrophage targeting moieties on the composition. The results are illustrated in FIG. 9. FIG. 9 is a dot plot showing the effect of concentration and macrophage targeting on the internalization of nanoparticles.

The addition of an internalizing antibody has the effect of lowering the dose of NP that need to be internalized. Targeting makes no difference at higher doses. Internalizing antibodies only affect the amount of particles internalized and NOT the internalization process itself.

In another assay, cells were incubated with the nanoparticles for 30 min, thoroughly washed and incubated in fresh media for 30 min. The results are shown in Table 1:

TABLE 1
Labeling Efficiency

| Rhodamine-labeled NPs | Labeling Efficiency |
|---|---|
| 1.0 mg/ml | 80% |
| 0.5 mg/ml | 60 |

### Example 4

### Macrophage Loading with Nanoparticles Does Not Impact Macrophage Function

Materials and Methods

An LDL uptake assay was used on bone marrow derived macrophages (MC) after loading with targeted or untargeted nanoparticles. Anti-F4/80 and anti-CD204 were used as targeting antibodies and LDL uptake was measured in a transwell assay. LDL was labeled with the flurophore DIL and Mean Fluorscence Intensity (MFI) after LDL uptake was measured before and after NP loading. B3Z are T cells used as negative controls

Results

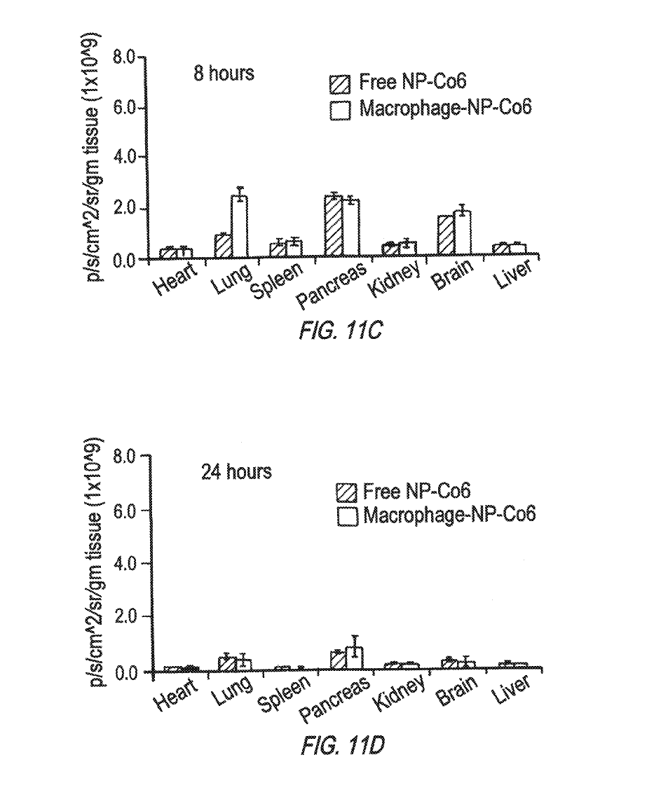

An LDL uptake assay was used to measure macrophage function without nanoparticles and after exposure to increasing concentrations of F4/80 and CD-204 targeted nanoparticles. The results are illustrated in FIGS. 10A and 10B, respectively.

FIGS. 10A-10B are bar graphs showing the effect of nanoparticle internalization on macrophage function (DIL-labeled LDL update measured as mean fluorescence intensity (MFI)) of B3Z T cells (negative control), untreated macrophage, and macrophage targeted with various concentrations of anti-F4/80 antibody (10A) and anti-CD204 antibody (10B).

### Example 5

### Biodistribution and Pharmacokinetics of Macrophage-Loaded Nanoparticles Versus Nanoparticles in the Living Animal

Materials and Methods

Cells were isolated from bone marrow of B6 mice and cultured in vitro with 10 ng/ml M-CSF and 1 ng/ml GM-CSF for 7 days prior to injection. BMDM cells were incubated with 4 mgs of particles for 2 hours. After 2 hours the media and the wash was collected and spin down at 10,000 rpm for 10 mins to collect the NPs.

A total of 15×10⁸ cells were resuspended in 3.7 ml PBS which contained all the free Co-6 particles from the media and the washes (NP-co6 concentration: 20 nmg/ml).

68 mg NP with Co-6 was dissolved in 3.4 ml PBS at concentration of 20 mg/ml.

Each mouse was iv injected with 200 μl.

After 1, 4, 8, 24 and 48 hours organs were dissected and stored at −40C until analysis.

Blood was collected by cardiac puncture. 200-300 μl blood was added into 400 μl RPMI 1640 media with 300 IU heparin for flow cytometry.

Experimental Set-Up

8×10⁶ cells in 0.2 ml PBS per mouse (1×10⁷ cells/mouse) via tail vein injection.

Blood and organs: heart, lung, spleen, pancreas, liver, kidney and brain was analyzed.

Cells in blood and splenocytes were analyzed by flow cytometry analysis.

Fluorescence intensity measured by IVIS imaging and verified by microplate reader after extraction.

### Groups:

PBS: 24hrs, n=3

Free Co-6 nanoparticles: 1, 4, 8, 24 and 48 hrs; n=3, 15 mice.

BMDM-Co-6 nanoparticles: 1, 4, 8, 24 and 48 hrs; n=3, 15 mice

Results

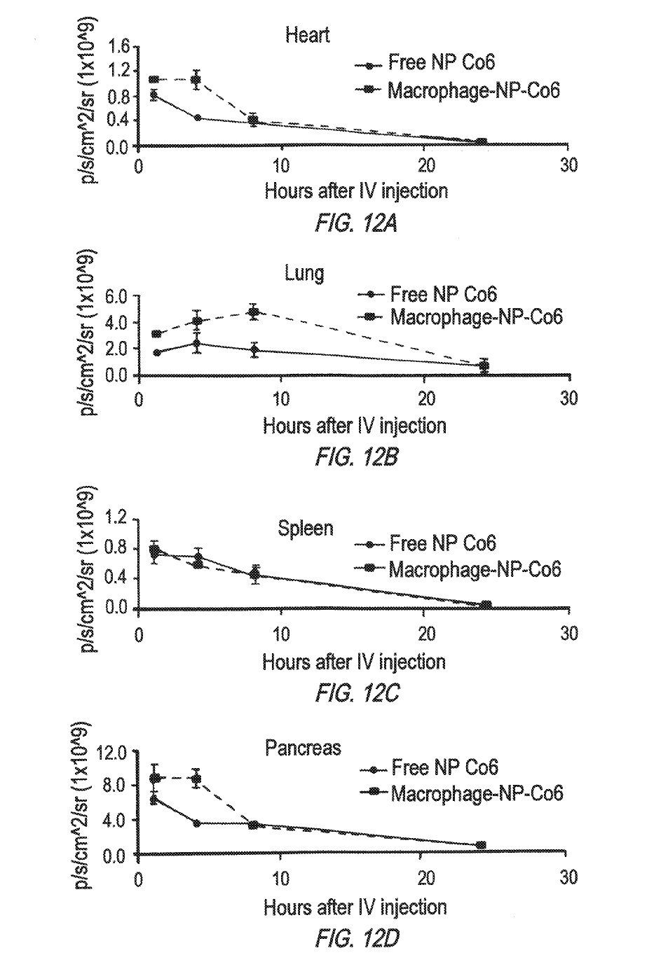

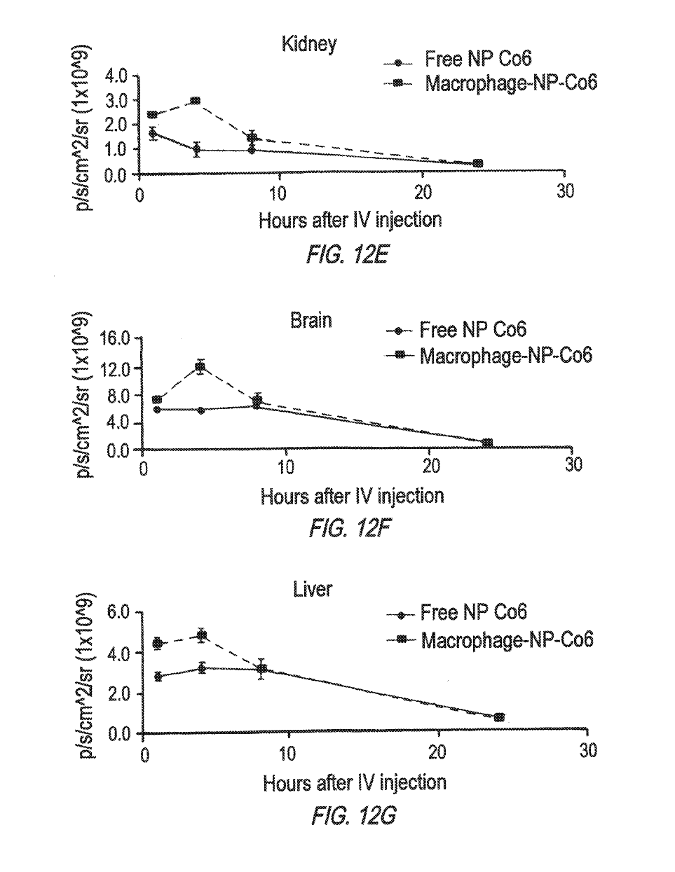

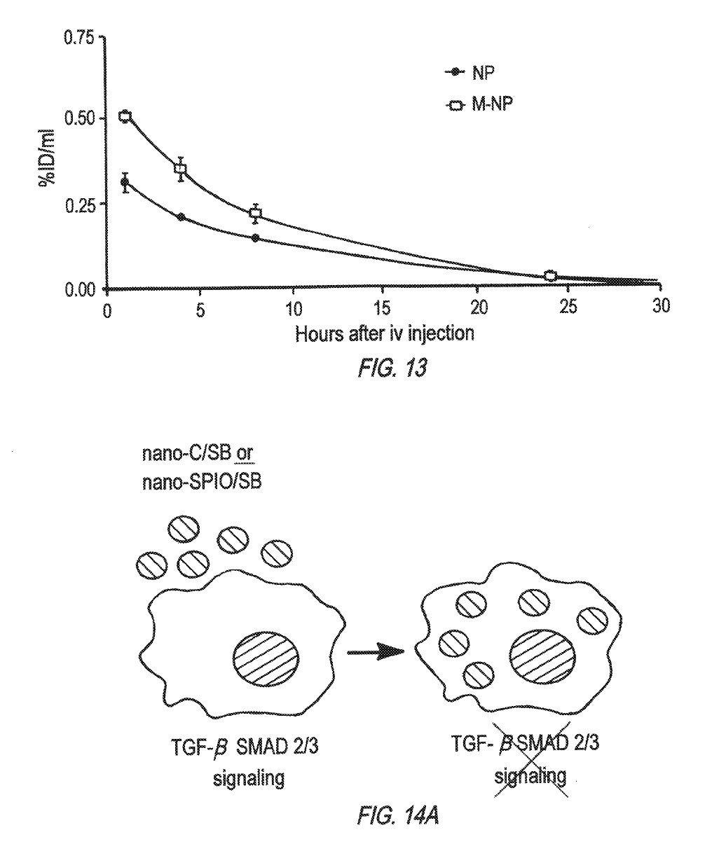

FIGS. 11A-11D are bar graphs showing the relative biodistribution (p/s/cm²/sr/gm tissue (1×10⁹)) of macrophage-loaded nanoparticles and free nanoparticles in the heart, lung, spleen, pancreas, kidney, brain, and liver in vivo, one (11A), four (11B), eight (11C), and twenty-four (11D) after intravenous administration. FIGS. 12A-12G are line graphs showing the relative biodistribution (p/s/cm²/sr (1×10⁹)) of macrophage-loaded nanoparticles and free nanoparticles in the heart (12A), lung (12B), spleen (12C), pancreas (12D), kidney (12E), brain (12F), and liver (12G) in vivo over the first 24 hours after intravenous administration. FIG. 13 is a curve showing the pharmacokinetics of Macrophage-loaded Nanoparticles (M-NP) and free nanoparticles (NP) in the blood over the first 24 hours after intravenous administration.

FIGS. 11A-12G illustrate the biodistribution of nanoparticles and macrophage-loaded nanoparticles in vivo. FIG. 13 illustrates the pharmacokinetic blood analysis of nanoparticles and macrophage-loaded nanoparticles in vivo.

### Example 6

### Nanoparticle Blockade of TGF-β Signaling in Peripheral Macrophages Mitigates Alzheimer-Like Pathology in TgF344-AD Rats

Transforming growth factor-beta (TGF-beta), an important immunoregulatory cytokine, has increased abundance in AD patient brains. Genetic ablation of TGF-beta-Smad 2/3 signaling in peripheral macrophages causes their brain recruitment and resolution of Alzheimer-like pathology including cerebral amyloidosis, which does not come at the cost of damaging neuroinflammation and actually restores cognitive function. Pharmacological blockade of TGF-beta signaling in peripheral macrophages using nanoparticles may be able to re-balance inflammation and mitigate AD-like pathology in the TgF344-AD rat model, which manifests the full spectrum of age-dependent AD pathologies and cognitive disturbance.

Materials and Methods

PEG-PLGA-COOH nanoparticles encapsulating SB505124 (a small molecule TGF-beta-Smad 2/3 inhibitor) (also referred to as “nano-SB505124”) and the nontoxic fluorescent tracker, Coumarin-6 (designated nano-C6/SB) superparamagnetic iron oxide (SPIO) were developed to specifically target peripheral macrophages as described above.

Two long-term peripheral treatment studies were performed with nano-C6/SB—one beginning treatment prior to plaque accumulation (e.g., 3 months—no plaque phenotype) and the other beginning after plaque accumulation (e.g., 8 month—moderate plaque phenotype). The drug dosage utilized is 2.5 mg/kg of SB inhibitor.

Results

Experiments were designed to determine whether pharmacological blockade of TGF-beta signaling in peripheral macrophages using next-generation nanoparticle technology can re-balance inflammation and mitigate AD-like pathology in the TgF344-AD rat model, which manifests the full spectrum of age-dependent AD pathologies and cognitive disturbance.

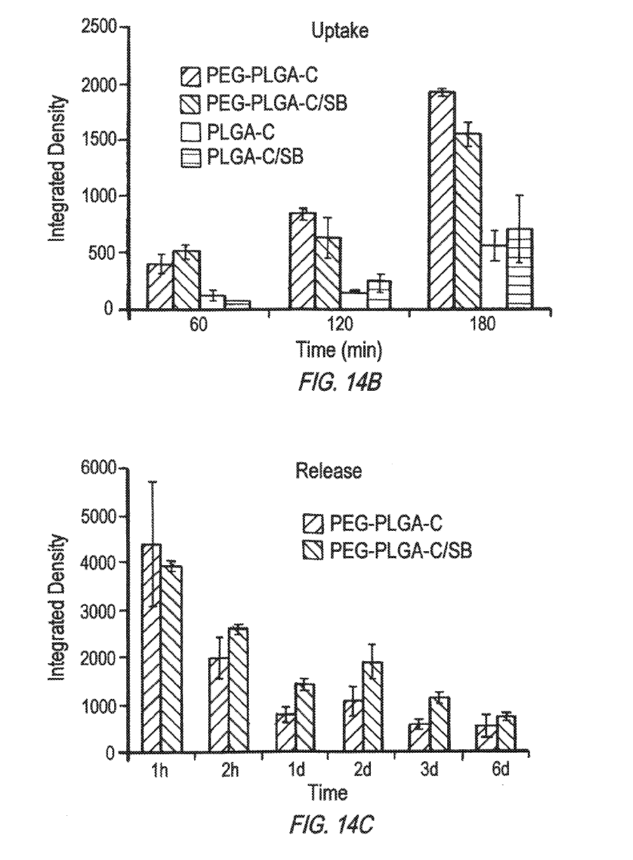

Results from in vitro experiments show that nano-C6/SB directly targets peripheral macrophages, effectively inhibiting TGF-beta signaling and increasing macrophage Abeta uptake. See FIGS. 14A, 14B and 14C. The results show selective drug delivery to peripheral macrophages using nanoparticles, and the addition of the PEG moiety to the COOH negative charged PLGA nanoparticles increases their bioavailability.

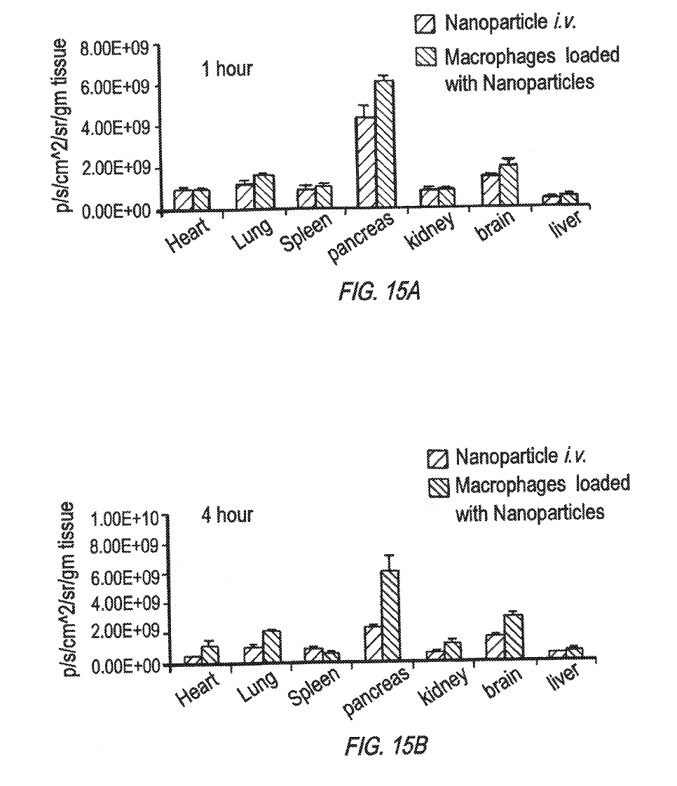

Tissue distribution over time following administration is shown in FIGS. 15A-15D (one, four, eight and 24 hours after administration). The results indicate that macrophage containing PEG-PLGA-COOH nanoparticles migrate to the CNS.

Confocal images of brain sections from a TgF344-AD rat showed Iba1⁺ cells, CD68⁺ phagolysosomes, and OC⁻ soluble fibrillar Aβ. FIG. 15E shows quantification of microglial phagolysosomes containing Aβ (n=6 cells) for mononuclear phagocytes distant from plaques (1) or associated with plaques (2). *p<0.01; by Student's t-test. These results demonstrate that, in principle, mononuclear phagocytes can phagocytose Aβ deposits in vivo.

Treatment of AD Model Rats with Nano-SB1947 Induces Migration of Macrophages to the CNS. Promotion of amyloid-β (Aβ) phagocytosis was identified in TgF344-AD rat brains after Nano-SB1947 treatment. Coumarin-6, a Nano-1947 surrogate marker, Iba1, CD68, and OC signal in and surrounding a mononuclear phagocyte found in the brain of a TgF344-AD rat treated with Nano-SB1947 shows peripheral macrophage uptake of Aβ following Nano-SB1947 treatment. Hippocampal confocal images of Nano-SB1947-treated TgF344-AD rats show CD11b-positive cells containing Coumarin-6 surrounding amyloid fibrils. FIGS. 15F and 15G show quantitative cell-specific analysis indicating Coumarin-6 signal is found in 10-35% of cells expressing Iba1, CD11b, or MHCII and not in cells expressing MAP2, GFAP, or Olig4 (ND=not detected) (15F) and that the vast majority of Coumarin-6-positive cells (80-90%) associate closely with amyloid plaques (15G).

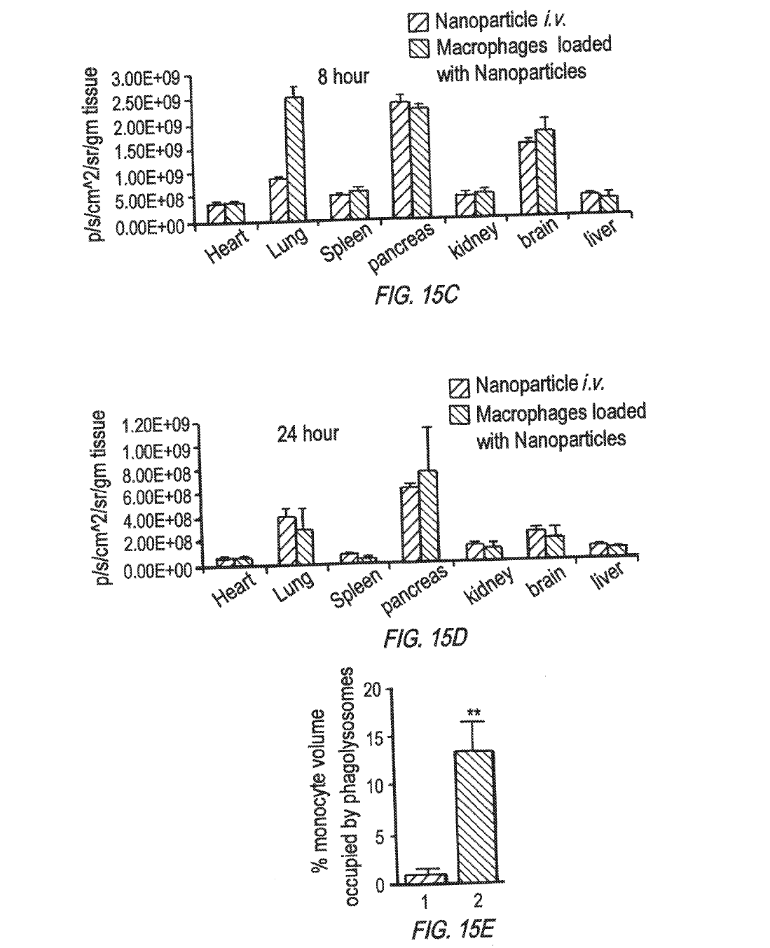

FIGS. 16A-16C show that Nano-SB1947 treatment reverses Aβ and tau aggregation and plaque formation.

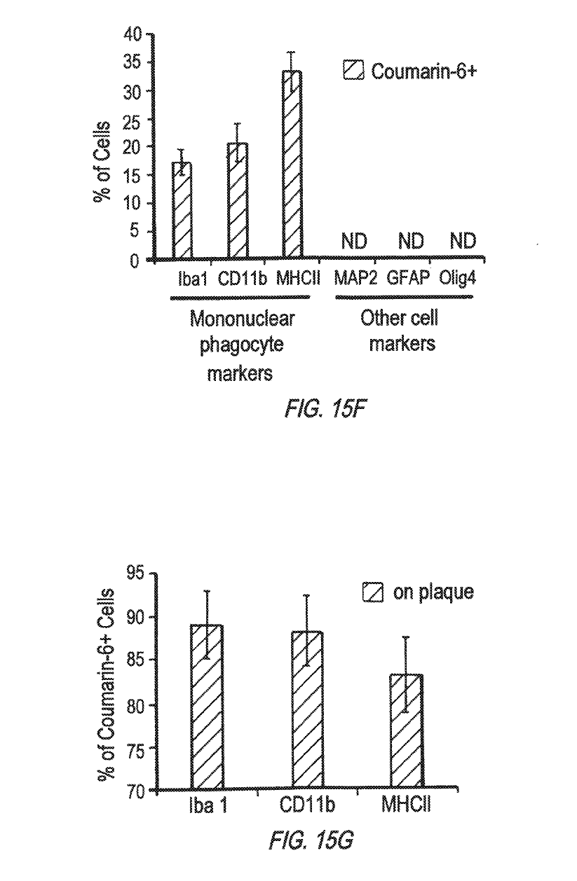

FIG. 17 shows an increase pre-synaptic density back to wildtype untreated rat levels, thus indicating a complete reversal of disease phenotype.

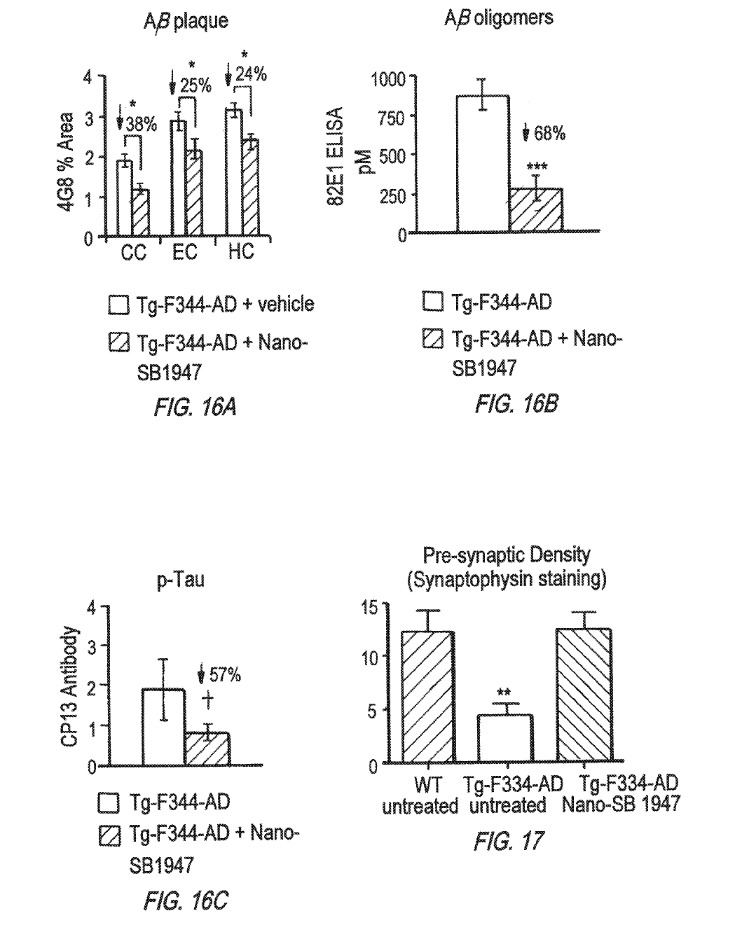

Following treatment, aged TgF344-AD rats and controls were behaviorally tested and their brains were analyzed for AD-like pathology. Results presented in FIGS. 18A and 18B show that Nano-SB1947 reverses behavioral defects in Tg-F344-AD rats using both the Novel Object Recognition Test and the Barnes Maze Test.

Peripheral nano-C6/SB treatment: 1) promotes brain infiltration of Coumarin-6-positive mononuclear phagocytes that localize to amyloid plaques; 2) attenuates cerebral amyloidosis and tauopathy; and 3) partially remediates cognitive deficits, with treatment regimen dominantly affecting outcome. The results show that PEG-PLGA-COOH nanoparticles encapsulating small molecule TGF-beta-Smad 2/3 inhibitors hold pre-clinical promise to directly target peripheral macrophages for cerebral amyloid clearance.

In the brain the cytokine TGF-β1 dampens microglial (macrophages in the brain) activation. Yet TGF-β1 overexpression promotes brain inflammation and the acceleration of vascular beta-amyloid (Aβ) deposits and neuronal amyloid peptide secretion. TGF-β1 belongs to the class of cytokines known as TGF-β, which are pleiotropic cytokines with central roles in immune suppression, immune homeostasis and repair after injury. Modulation of TGF-β in the body requires precise and localized dose delivery.

Blocking innate immune TGF-β signaling leads to brain infiltration of peripheral macrophages and beneficial cereberal amyloid aggregate clearance. The results discussed above exemplify compositions and methods for enhancing macrophage uptake of nanoparticles loaded with TGFβ antagonists. This is not only important for amyloidosis (Alzheimer's) therapy, but also for a number of applications ranging from inflammation to cancer.

The results show that efficient clearance of Amyloid-β (Aβ) plaques relies on the efficiency of NP uptake by peripheral macrophages. Generally, macrophages are, “first responders” in many disease states. They are the primary immune cells that respond to an infection, wound or any bodily injury. As such, macrophages alert the immune system (both adaptive and innate) to the nature of dysfunction. Macrophages, therefore, can be used to deliver immunomodulatory agents to the site of infection, inflammation, tumor growth or, as exemplified above, delivery of TGFβ1 antagonists that reduce plaque aggregates in the brain.

Given that macrophages are adept at clearing particulate matter, such as bacteria, viruses, parasites, cell debris and even peptide aggregates, the macrophages can be used as carriers, “Trojan horses,” to position the dose of TGF-β1 antagonists loaded in nanoparticles to cerebral plaque regions in the brain. TGF-β is a pleiotropic cytokine.

The results demonstrate that the compositions and methodology are useful for enhancing macrophage uptake of nanoparticles loaded with a variety of agents (therapeutic, immunomodulatory drugs, invasive and non-invasive imaging agents, with application in therapy as well as diagnostics or imaging of the Aβ accumulation in the brain). Enhancing uptake is important because uptake efficiency is related to therapeutic efficiency needed for plaque dissolution.

The results also demonstrate the efficacy of TGF-β pathway inhibitor loaded NP and uptake by peripheral macrophages for treatment of rat models of Aβ aggregates in the brain. TGF-β1 in the brain dampens neuroinflammation, and its overexpression promotes brain inflammation and can accelerate Vascular β-amyloid deposits and elicit neuronal amyloid peptide secretion. Thus, blocking innate immune TGF-β signaling leads to brain infiltration of peripheral macrophages and beneficial cerebral amyloid aggregate clearance.

### Example 7

### Quantitative 3D in Silico Modeling (q3DISM) of Cerebral Amyloid-βPhagocytosis in Rodent Models of Alzheimer's Disease

Alzheimer's disease (AD), the most common age-related dementia, is characterized by cerebral amyloid-β (Aβ) accumulation as “senile” β-amyloid plaques, chronic low-level neuroinflammation, tauopathy, neuronal loss, and cognitive disturbance. In AD patient brains, neuroinflammation is earmarked by reactive astrocytes and mononuclear phagocytes (referred to as microglia, although their central vs. peripheral origin remains unclear) surrounding amyloid deposits. As the innate immune sentinels of the CNS, microglia are centrally positioned to clear brain Aβ. However, microglial recruitment to β-amyloid plaques is accompanied by very little, if any, Aβ phagocytosis. One hypothesis is that microglia are initially neuroprotective by phagocytozing small assemblies of Aβ. However, eventually these cells become neurotoxic as overwhelming Aβ burden and/or age-related functional decline provokes microglia into a dysfunctional pro-inflammatory phenotype, contributing to neurotoxicity and cognitive decline.

Recent genome-wide association studies (GWAS) have identified a cluster of AD risk alleles belonging to core innate immune pathways that modulate phagocytosis. Consequently, the immune response to cerebral amyloid deposition has become a major area of interest, both in terms of understanding AD etiology and for developing new therapeutic approaches. Yet, there is a vital need for methodology to evaluate Aβ phagocytosis in vivo.

As discussed in more detail below, to address this need, quantitative 3D in silico modeling (q3DISM) has been developed to enable true 3D quantitation of cerebral AB phagocytosis by mononuclear phagocytes in rodent models of Alzheimer-like disease. Limited only by the extent to which they recapitulate disease, animal models have proven invaluable for understanding AD pathoetiology and for evaluating experimental therapeutics. Owing to the fact that mutations in the presenilin (PS) and amyloid precursor protein (APP) genes independently cause autosomal dominant AD, these mutant transgenes have been extensively used to generate transgenic rodent models. Transgenic APP/PS1 mice simultaneously co-expressing “Swedish” mutant human APP (APP_{swe}) and Δ exon 9 mutant human presenilin 1 (PS1ΔE9) present with accelerated cerebral amyloidosis and neuroinflammation. Bi-transgenic rats co-injected with APP_{swe} and PSΔE9 constructs (line TgF344-AD, on a Fischer 344 background) were also developed. Unlike transgenic mouse models of cerebral amyloidosis, TgF344-AD rats develop cerebral amyloid that precedes tauopathy, apoptotic loss of neurons, and behavioral impairment.

A protocol for immunostaining microglia, phagolysosomes and Aβ deposits in brain sections from APP/PS1 mice and TgF344-AD rats, and acquisition of large z-dimensional confocal images, was developed and utilized for in silico generation and analysis of true 3D reconstructions from confocal datasets allowing quantitation of Aβ uptake into microglial phagolysosomes. The methodology can be used to quantify virtually any form of phagocytosis in vivo.

Materials and Methods

Statement of Research Ethics

All experiments involving animals detailed herein were approved by the University of Southern California Institutional Animal Care and Use Committee and performed in strict accordance with National Institutes of Health guidelines and recommendations from the Association for Assessment and Accreditation of Laboratory Animal Care International.

Rodent Brain Isolation and Preparation for Immunostaining

Day 1:
1.1) Place aged TgF344-AD rats (14 month-old) or APP/PS1 mice (12 month-old) under continuous deep isoflurane anesthesia (4%). Assess the depth of anesthesia by toe pinch and the absence of withdrawal reflex.
1.2) Cut through both sides of the rib cage and lift to expose the heart. Insert a 23 gauge (23G) needle into the left ventricle of the heart and make a small incision into the right atrium. Proceed to exsanguination by transcardial perfusion with ice-cold phosphate-buffered saline (PBS) using a peristaltic pump (30 ml for mice, 150-200 ml for rats).
1.3) Make a caudal midline incision into the skin and move the skin and muscle aside. Cut through the top of the skull along the midline and between the eyes. Remove the bone plates and isolate the whole brain from the skull.
1.4) Place the brain into a coronal rodent brain matrix and slice it into quarters. Incubate posterior quarters overnight (16 h) in paraformaldehyde fixative (4% PFA in PBS) at 4° C. Wash three times in PBS, and then transfer to 70% ethanol. Caution: PFA is toxic and should be handled under a chemical hood with appropriate personal protection equipment.

Day 2:
1.5) Place brain quarters into embedding cassettes and progressively dehydrate tissue in successively more concentrated 1 h ethanol baths (70%, 80%, 95%, and 100% ×3).
1.6) Clear ethanol from the tissue with three successive 100% xylene baths (1 h each). Caution: Xylene is toxic and should be handled under a chemical hood with appropriate personal protection equipment.
1.7) Embed tissue in paraffin blocks after two molten paraffin wax baths (56-58° C., 90 min each).

Day 3:
1.8) Cut 10 μm-thick sections of paraffin-embedded brains using a microtome. Dip sections into a water bath (50° C. for 1 min) and apply to microscope slides. Leave the slides to dry overnight, ensuring tissue adhesion to the slide.

Immunostaining

Different combinations of antibodies can be utilized for the staining procedure described below.

Day 4:
2.1) De-paraffinize brain sections using two 100% xylene baths (12 min each).
2.2) Rehydrate brain sections in successive ethanol baths—100% for 10 min, 95% for 5 min, 80% for 10 min, and finally 70% for 15 min—followed by 3× PBS washes [5 min at room temperature (RT), with light agitation]. Meanwhile, heat antigen retrieval solution to 95-97 DC on a hot plate with magnetic bar stirring.
2.3) Incubate brain sections in antigen retrieval solution at 95-9rC for 30 min. Then, wash 3× in PBS (5 min at RT, with light agitation).
2.4) Quickly dry slides using delicate task wipers to avoid tissue drying, and draw a hydrophobic barrier around the tissue area with a hydrophobic barrier pen. Fill the encircled tissue region with blocking buffer [PBS containing 0.3% Triton X-100 and 10% Normal Donkey Serum (NDS)] and incubate at room temperature for 1 h in a humidified chamber.
2.5) Replace blocking buffer with Iba1 primary antibody (diluted in blocking buffer) to label mononuclear phagocytes, and incubate overnight at 4 DC in a humidified chamber. For antibody hosts and working dilutions.

Day 5:
2.6) Rinse primary antibody with 3× PBS baths (5 min at RT with light agitation). Incubate with fluorescent secondary antibody (conjugated with a 594 nm emission fluorophore) for 1 h (in blocking buffer at RT in the dark) followed by 3× PBS baths (5 min at RT with light agitation). At this time, maintain sections in the dark to avoid fluorescent signal bleaching.

Day 6-7:
2.7) Repeat steps 2.5 & 2.6 with CD68 (rat brains) or LAMP1 (mouse tissue) antibodies and appropriate secondary antibodies (coupled with a 488 nm emission fluorophore) to label phagolysosomes.

Day 7-8:
2.8) Repeat steps 2.5 & 2.6 with OC (rat tissue) or 4G8 (mouse brains) antibodies and appropriate secondary antibodies (coupled to a 647 nm fluorophore) to label Aβ deposits. Note: Alternatively, 6E1D antibody can be used successfully both on mouse and rat tissue.
2.9) Allow sections to completely dry overnight at RT in the dark. Then, cover specimens with a cover slip sealed by fluorescence mounting media containing DAPI.

Acquisition of Large Z-Stack Confocal Data Sets

This protocol utilizes a fully automated laser scanning confocal microscope equipped with a 60× objective and 405 nm, 488 nm, 594 nm, and 647 nm lasers. All equipment is computer controlled by imaging and laser control software. Prior to beginning the imaging protocol, power on the computer, epifluorescent lamp, microscope, lasers and camera.

Day 9:
3.1) Select the 60× microscope objective. Add immersion oil to the lens, and place the sample onto the microscope stage slide holder. Raise the objective until the oil makes contact with the slide. Adjust the focal plane to locate amyloid plaques in the hippocampus or cerebral cortex using epifluorescent illumination through the oculars.
3.2) Acquire confocal images of activated mononuclear phagocytes surrounding amyloid deposits in the hippocampus or cortex of rodents by confocal microscopy (60× magnification, zstack steps: 0.25 μm<z<0.40 μm, number of steps 25<n<35).

q3DISM

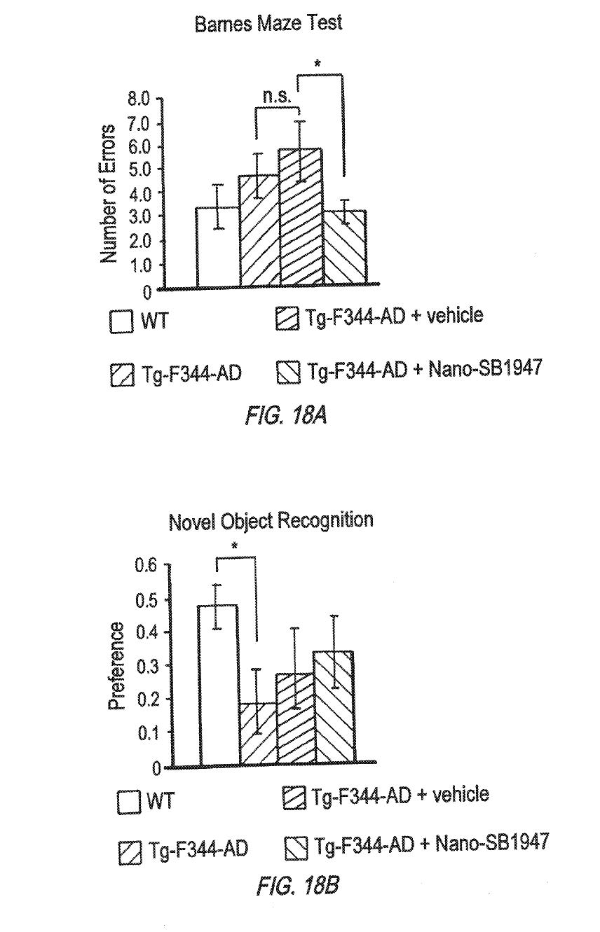

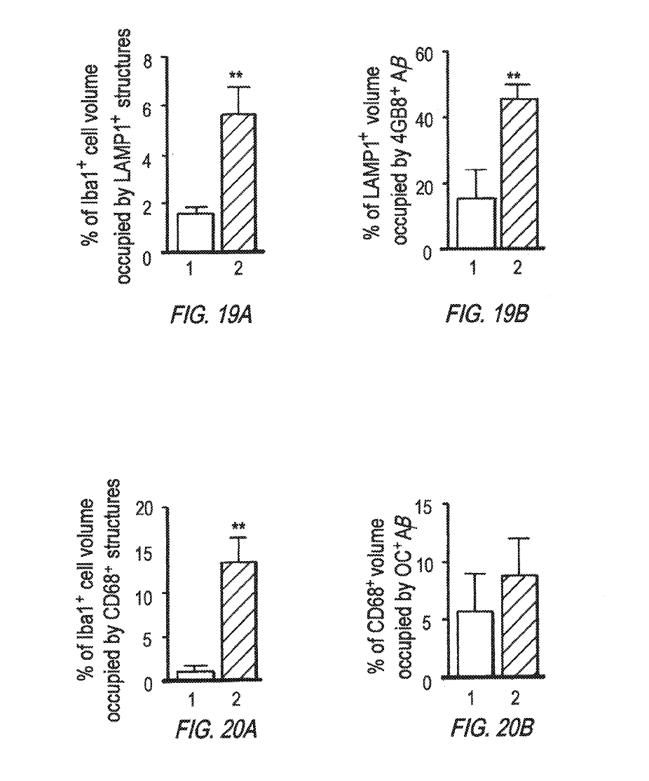

In order to yield significant results, analyzing a minimum of 3 images per animal/region of interest is generally preferred. For each image, the abundance of cells to analyze may vary depending on experimental paradigms. In the representative results discussed below, 3 cells per condition were analyzed (e.g., mononuclear phagocytes distant from or associated with plaques; see FIGS. 19A-19B and 20A-20B).

Day 10:
4.1) Analyze confocal datasets with scientific 3D image processing and analysis software co-localization (coloc) module for spatial proximity of Iba1/CD68 (rat tissue) or Iba1/LAMP1 (mouse tissue) staining in all z-planes simultaneously. Create Iba⁺/CD68⁺ or Iba⁺/LAMP1⁺ colocalization channels that correspond to phagolysosomes within activated mononuclear phagocytes.
4.1.1) Select TRITC for channel A (corresponding to Iba1 staining coupled with 594 nm fluorophore) and FITC for channel B (corresponding to C068 or LAMP1 staining coupled with 488 nm fluorophore). On the right-hand side of the software window for ‘mode check,’ select ‘threshold,’ and for ‘coloc intensities,’ select ‘source channels’.
4.1.2) Click ‘Edit’ to select ‘coloc color’ on the right-hand side of the software window.
4.1.3) For each channel independently, adjust thresholds to include specific staining and exclude background/non-specific signals. Once adjusted, do not change thresholds between images to ensure an unbiased analysis. The colocalized voxels (pixels from all z-stacks) will appear in the color selected in step 4.1.2 in all z-stacks simultaneously.
4.1.4) Click ‘build coloc channel’. The colocalization channel created will appear in the display adjustment window.
4.1.5) Click on the coloc channel to open channel statistics. The ‘% of volume/material A above threshold colocalized’ represents the % of Iba1 signal (voxels corresponding to the 594 nm fluorophore) colocalized with LAMP1 or CD68 signal (voxels corresponding to the 488 nm fluorophore). More simply, this is the monocyte volume occupied by phagolysosomes (see FIGS. 19A and 20A).

‘% of volume/material B above threshold colocalized’ corresponds to % of LAMP1 or CD68 signal colocalized with Iba1. This should be close to 100%, as phagolysosomes are intracellular structures. Values are based on ratio of signal from all z-stacks above threshold colocalized.
4.2) Using the coloc module, analyze the coloc channel created in step 4.1 for spatial proximity with OC (rat tissue) or 4G8 (mouse tissue) A˜ signals. This allows for quantitation of Aβ encapsulated within phagolysosomes.
4.2.1) Select the channel A coloc dataset (corresponding to the Iba1/C068 or Iba1/LAMP1 coloc channel created in step 4.1) and Cy5 for channel B (corresponding to OC or 4G8 staining coupled with 647 nm fluorophore).
4.2.2) Build a coloc channel as described in steps 4.1.2 to 4.1.5.
4.2.3) Click on the coloc channel to open channel statistics. The ‘% of volume/material A above threshold colocalized’ represents the % of Iba1/LAMP1 or Iba1/C068 (voxels corresponding to the coloc channel built in step 4.1.5.) colocalized with OC or 4G8 signal (voxels corresponding to the 647 nm fluorophore). This is the phagolysosomal volume occupied by Aβ.

‘% of volume/material B above threshold colocalized’ corresponds with % of total Aβ signal colocalized with phagolysosomes. This can be used to evaluate the fraction of total Aβ deposits encapsulated within phagolysosomes (not shown in the representative results).
4.3) Use the surpass module to reconstruct the confocal image stacks and generate 3D models of Aβ encapsulated within monocyte phagolysosomes.
4.3.1) In the ‘display adjustment’ window, select TRITC (to show only Iba1 staining). In the ‘volume properties’ window, click ‘add new surface’.
4.3.2) In step ‘1/5 algorithm’, in ‘settings,’ select ‘surface.’ Also, in ‘color,’ select color type in palette or RGB and adjust transparency.
Check box ‘select region of interest’. Click next.
4.3.3) In Step ‘2/5 region of interest,’ draw a window around the cell of interest by adjusting x, y and z coordinates. Click next.
4.3.4) In Step ‘3/5 source channel’, select the TRITC source channel. Check the box ‘smooth,’ and set surface area detail level to 0.4 !-tm. For ‘thresholding,’ select absolute intensity. Click next.
4.3.5) In Step ‘4/5 threshold,’ adjust threshold so that the volume created overlaps perfectly with the TRITC channel signal. Click next.
4.3.6) In Step ‘5/5 classify surfaces,’ in section ‘filter type,’ select objects depending on their size to be included or excluded from the volumes to be created. Click finish, execute all creation steps, and exit the wizard.
4.3.7) Repeat steps 4.3.1 to 4.3.6 to create a 3D surface for the FITC channel (LAMP1+ or CD68+ phagolysosomes).
4.3.8) Repeat steps 4.3.1 to 4.3.6 to create a 3D surface for the Cy5 channel (OC+ or 4G8+ Aβ deposits).

Representative Results:

Using the mUlti-stage methodology for q3DISM detailed above, Aβ uptake into monocyte phagolysosomes in the brains of APP/PS1 mice (FIG. 19A-19B) and TgF344-AD rats (FIG. 20A-20B) was quantified. Therefore, the q3DISM methodology allows for analysis of mononuclear phagocytes in mouse and rat models of AD.

The volume occupied by CD68+ phagolysosomes is significantly increased in Iba1⁺ mononuclear phagocytes associated with compared to distant from plaques both in APP/PS1 mice (FIGS. 19A and 19B) and TgF344-AD rats (FIGS. 20A and 20B). These data show that mononuclear phagocytes near plaques are poised for phagocytosis compared to cells located away from the plaque. In order to illustrate the range of data from staining different Aβ conformers, various antibodies in mouse and rat tissue were used. Directly comparing mouse and rat models of AD with regards to Aβ phagocytosis was not necessarily desired. Nonetheless, it was noteworthy that plaque associated mononuclear phagocytes had increased Aβ uptake in APP/PS1 mice compared to cells distant from plaques (FIG. 19B). TgF344-AD rats had very modest uptake of Aβ fibrils overall, and no change in Aβ fibril phagocytosis as a function of distance from plaques (FIG. 20B).

The protocol described above provided for true 3D quantitation of Aβ phagocytosis in vivo by mononuclear phagocytes. It relies on specific labeling of cellular and subcellular compartments as well as Aβ deposits. Specifically, Iba1 (Ionized calcium binding adaptor molecule 1), a protein that is involved in membrane ruffling and phagocytosis upon cell activation was used to stain cerebral mononuclear phagocytes. While Iba1⁺ cells are generally regarded as brain-resident microglia, it remains possible that peripherally infiltrating mononuclear phagocytes comprise a subset of immunolabeled cells. Intracellular phagolysosomes are revealed with antibodies recognizing members of the lysosomal/endosomal-associated membrane glycoprotein (LAMP) family: LAMP120 or CD6821. The latter is primarily localized to lysosomes and endosomes, with a smaller fraction circulating to the cell surface. LAMP1 and CD68 antibodies have both been successfully used in rat and mouse tissue, and the decision on which to use is primarily dependent on the host of other antibodies used for co-staining. Various antibodies can be used for detection of Aβ. In mouse tissue, 4G8 (recognizing human and mouse amino acid residues 17-24 of β-amyloid peptide, FIG. 19A-19B) and 6E10 (recognizing human amino acid residues 1-17) have been used successfully, allowing detection and quantitation of Aβ content in LAMP1⁺ or CD68⁺ phagolysosomes present in Iba1+ mononuclear phagocytes. Using the q301SM methodology, increased Aβ load was observed in Iba1⁺ cells associated with global reduction of amyloid load in brains of APPPS1/IL-10^{-/-} mice, whereas others showed decreased Aβ uptake into Iba1⁺ cells accompanied by increased amyloid load and plaque volume in brains of Rag-5xfAD mice. These results indicate that the phenomenon observed is a “snapshot” of active Aβ phagocytosis, although this methodology cannot be used to predict if the phagocytosed material is efficiently digested and cleared.

In rat tissue, Aβ deposits were detected using OC (recognizing soluble Aβ42 oligomers/fibrils, FIG. 20A-20B) or 6E1017 (not shown). Interestingly, very little OC⁺ fibrils were present in monocyte phagolysosomes in TgF344-AO rat brains. This phenomenon has previously been reported in APP23 transgenic mice using immunogold staining and 3D. A variety of antibodies for detection of different Aβ species/conformers are available and can be utilized in the disclosed protocol.

The q3DISM technique has been adopted by us and others to quantify Aβ phagocytosis in the context of AD. However, the method can be adapted to evaluate virtually any form of phagocytosis. It may also be applied to tissues other than the brain, and to other animal species (including humans). The procedures described in this protocol rely on standard immunostaining, detected with fluorescence and imaged with a confocal microscope. In the above experiments, paraffin-embedded tissue was prepared according to guidelines provided by the antibody manufacturers. The present protocol can also be adapted for use with fresh tissue (embedded in 2% agarose in PBS). The protocol can also be adapted to detect phagocytosis of co-aggregating proteins. This is made possible by staining and acquiring images composed of more than 4 fluorescent colors. In this case, the creation of the first colocalization channel (monocyte/phagolysosome) would be followed by creation of a second colocalization channel (aggregated protein 1/co-aggregated protein 2). Then, the two channels would be used for 3D analysis and modeling.

The q3DISM technique is constrained principally by the specificity and sensitivity of immunostaining, quality of antibodies, and colocalization approach. This multi-step procedure has a few potential dangers that can affect quality of the staining and, therefore, 3D quantitation of Aβ uptake. The first important step of the procedure is the isolation of rodent brains. Indeed, careful tissue handling is fundamental to avoid damage to the brain and to ensure that brain structures remain intact when sectioned. Second, tissue fixation time is quite important since over-fixation (more than 16 h in 4% PFA) limits detection of subcellular phagolysosomes and Aβ content. The successive staining of 1) monocytes, 2) phagolysosomes and 3) Aβ is important for the success of the method. It is important to use the antibodies consecutively—not concomitantly. Additionally, acquisition of the z-stack confocal images is important, because 3D modeling of cells, phagolysosomes and Aβ peptide as well as colocalization analyses depend on the quality of imaging of cellular and subcellular structures through the thickness of the tissue section. Finally, adjustment of thresholds in the software is crucial for 3D modeling and for colocalization analysis. Indeed, thresholds for the different channels need to be carefully chosen, since they will determine which signal is included (specific) or excluded (background) from the analysis. It is also fundamental that the thresholds chosen remain consistent between samples in the case of the comparison of different animals/specimens. So far, classical immunohistostaining, magnetic resonance imaging and positron emission tomography have been the imaging methods of choice for in vivo visualization and quantitation of β-amyloid. Although highly useful for large-scale quantitation of amyloid burden in brains of rodents or AD patients, these methods are inefficient for visualization of intracellular Aβ. Other techniques using confocal microscopy, fluorescence spectroscopy and flow cytometry exist and have been used by us and others to discriminate and quantify intracellular β-amyloid content in vitro. Immunogold staining coupled with 3D reconstruction was previously used to highlight the absence of amyloid fibrils within microglia in vivo in APP23 transgenic mice, and another study successfully used confocal imaging and 3D surface reconstruction of microglia associated with β-amyloid plaques to show limited Aβ interaction and uptake. However, these techniques do not allow for quantitation of intra-phagolysosomal Aβ species. A fluorescence-based in vivo assay was also recently developed, allowing the measurement of phagocytic activity by peripheral macrophages in the rat. This method allows for quantitation of fluorescent bioprobes in phagolysosomes in vivo, however, data available so far are limited to the periphery.

See also Guillot-Sestier, et al., “Quantitative 3D In Silico Modeling (q3DISM) of Cerebral Amyloid-beta Phagocytosis in Rodent Models of Alzheimer's Disease,”J. Vis. Exp. (118), e54868, doi:10.3791/54868 (2016), which is specifically incorporated by reference herein in entirety.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A nanoparticulate composition for selective delivery to macrophage or other phagocytic cells comprising:
nanoparticles selected from the group consisting of polymeric nanoparticles, liposomes, micelles and vesicles, having a negative surface charge,
the nanoparticles comprising therapeutic, prophylactic and/or diagnostic agent,
wherein the negatively charged nanoparticles are internalized by macrophages and other phagocytic cells.

2. The composition of claim 1 wherein the nanoparticles are polymeric nanoparticles.

3. The composition of claim 1, wherein the negative charge of the nanoparticles is effective to (1) increase circulation in subject following systemic administration, (2) increase internalization by macrophage or other phagocytic cells, (3) increase release within the macrophage or other phagocytic cells, or (4) a combination thereof, relative to charge-neutral or charge-positive nanoparticles.

4. The composition of claim 1, wherein the zeta potential of the nanoparticles is between about −100 mV and about −1 mV.

5. The composition of claim 1, wherein the zeta potential is between about −20 mV and about −1 mV.

6. The composition of claim 1, wherein the nanoparticles are polymeric particles comprising polyesters.

7. The composition of claim 6, wherein the nanoparticles are polymeric nanoparticles comprising a polymer having the structure A-X where A is a hydrophobic molecule or hydrophobic polymer, and X is a terminal moiety that imparts a negative charge to the particle.

8. The composition of claim 6, wherein the nanoparticles are polymeric nanoparticles comprising the structure A-B-X where A is a hydrophobic molecule or hydrophobic polymer, B is a hydrophilic molecule or hydrophilic polymer, and X is a terminal moiety that imparts a negative charge.

9. The composition of claim 8, wherein the biodegradable is conjugated to a polyalkene oxide or block copolymer thereof which is conjugated to a negatively charged terminal moiety.

10. The composition of claim 6, wherein the polyester is poly(lactic acid), poly(glycolic acid), poly(lactic-co-glycolic acid), or a blend or copolymer thereof.

11. The composition of claim 1, wherein the nanoparticles are liposomes comprising an anionic lipid; a terminal moiety that imparts a negative charge attached to a cationic, neutral lipid, an anionic lipid, and/or to a polyalkene oxide or block copolymer thereof; or a combination thereof.

12. The composition of claim 7, wherein the terminal moiety is an acidic group or an anionic group.

13. The composition of claim 12, wherein the acidic group is selected from carboxylic acids, protonated sulfates, protonated sulfonates, protonated phosphates, singly- or doubly protonated phosphonates, and singly- or doubly protonated hydroxamate.

14. The composition of claim 13, wherein the anionic group is selected from carboxylates, sulfates, sulfonates, singly- or doubly deprotonated phosphate, singly- or doubly deprotonated phosphonate, and hydroxamate.

15. The composition of claim 13, wherein the acidic group is COOH.

16. The composition of claim 1 further comprising a macrophage targeting moiety bound to the nanoparticles

17. The composition of claim 1 further comprising a cation in an effective amount to reduce the negative charge of the particles.

18. The composition of claim 17, wherein the cation is Ca2+ or Mg2+.

19. The composition claim 1, wherein the agent is selected from the group consisting of small molecules, peptides, polypeptides, proteins, nucleic acids, lipids, saccharides or polysaccharides, and combinations thereof.

20. The composition of claim 19 wherein the agent is an immunomodulatory agent.

21. The composition of claim 19 wherein the agent is a TGF-β inhibitor or an anti-inflammatory agent.

22. The composition of claim 21, wherein the TGF-beta inhibitor is a TGF-beta receptor inhibitor.

23. The composition of claim 21, wherein the TGF-beta inhibitor is an Alk5 inhibitor.

24. The composition of claim 21, wherein the TGF-beta inhibitor is a TGF-beta type 1 receptor

25. The composition of claim 21, wherein the TGF-beta inhibitor is a TGF-beta type II receptor

26. The composition of claim 19, wherein the agent is a diagnostic agent.

27. A method of treating or diagnosing a subject in need thereof comprising administering to the subject an effective amount of the composition of claim 1.

28. The method of claim 27, wherein the subject has an infection or a tumor in the brain or central nervous system.

29. The method of claim 27, wherein the subject has an inflammatory disorder, an immune disease, or a neurodegenerative disease.

30. The method of claim 27, where the subject has a Protein Misfolding Disorder.

31. The method of claim 27, wherein the subject has Alzheimer's disease, Type II diabetes, atherosclerosis, cardiovascular disease, or immune disease or disorder.

32. The method of claim 27, wherein the subject has a disease or disorder characterized by misfolded proteins.

33. The method of claim 27, wherein the subject has a prion disease, Lewy Body Dementia (LBD), Parkinson's disease (PD), Alzheimer's disease (AD), or amyotrophic lateral sclerosis (ALS).

34. The method of claim 27, wherein the subject has brain cancer.

35. The method of claim 34, wherein the brain cancer is medulloblastoma or glioblastoma muliforme.

36. The method of claim 27, wherein the subject has or is in danger of developing pathological protein aggregates.

37. The method of claim 36, wherein the pathological protein aggregates comprise Abeta, tau, or alpha-synuclein.

38. The method of claim 27, wherein the composition is administered parenterally.

39. The method of imaging a subject of claim 27 comprising administering to a subject the composition wherein the agent is an imaging agent, and acquiring at least one image of at least a portion of the subject.

40. The method of claim 39, wherein the imaging agent is MRI-based tracers.

41. The method of claim 40, wherein the magnetic resonance imaging (MRI)-based tracer is paramagnetic, superparamagnetic or protein-based.

42. The method of claim 41, wherein the superparamagnetic tracer is iron oxide or iron platinum.

43. The method of claim 39, wherein the imaging agent is a Computed tomography (CT) tracer.

44. The method of claim 43, wherein the tracer is radioactive.

45. The method of claim 43, wherein the tracer is not radioactive.
